(19) United States
(12) Patent Application Publication
(10) Pub. No.: US 2011/0295006 A1
(43) Pub. Date: Dec. 1, 2011

(54) CYCLIC DIPEPTIDES AS FEED ADDITIVES

(75) Inventors: Christoph KOBLER, Alzenau (DE); Thomas HAEUSSNER, Bad Orb (DE); Christoph WECKBECKER, Gruendau-Lieblos (DE)

(73) Assignee: EVONIK DEGUSSA GMBH, Essen (DE)

(21) Appl. No.: 13/116,823

(22) Filed: May 26, 2011

Related U.S. Application Data
(60) Provisional application No. 61/349,548, filed on May 28, 2010.

(30) Foreign Application Priority Data
May 27, 2010 (DE) 102010029399.7

(51) Int. Cl. C07K 5/12

(52) U.S. Cl. 544/357; 544/383

(57) ABSTRACT

Feed additives containing essential amino acids which are diketopiperazines of formulas (IV) or (V) or salts thereof are provided:

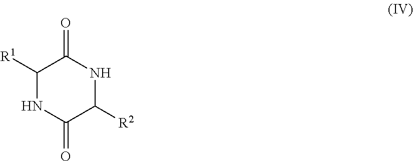

In formulas (IV) and (V), R¹ and R² may be an amino acid residue such as methionine, lysine, threonine, tryptophan, histidine, valine, leucine, isoleucine, phenylalanine, arginine, and cysteine, and may optionally be the same residue.

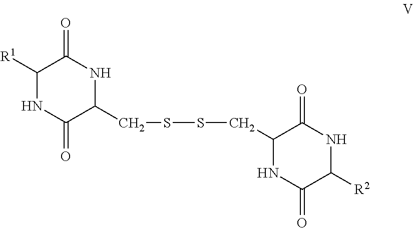

Additionally provided are the diketopiperazines of formulas (IV) and (V) and a method to for their production.

### CROSS REFERENCE TO RELATED APPLICATIONS

This application claims priority to German Application No. 102010029399.7, filed May 27, 2010 and U.S. Provisional Application No. 61/349,548, filed May 28, 2010, the disclosures of which are incorporated herein by reference in their entireties.

### BACKGROUND OF THE INVENTION

1. Field of the Invention

The present invention relates to feed additives containing chemically protected dipeptides in the form of diketopiperazines (cyclo-dipeptides, dehydrodipeptides) of essential, limiting amino acids, e.g. methionine, lysine, threonine, tryptophan, cysteine and cystine, and synthesis and use thereof in feeds for feeding ruminants and especially fish and crustaceans in aquaculture.

2. Description of the Background

The essential amino acids (EAA) methionine, lysine, threonine, tryptophan, histidine, valine, leucine, isoleucine, phenylalanine, arginine, cysteine and cystine are very important constituents in feeds and play an important role in the economic rearing of livestock, e.g. chicken, pigs, ruminants, and in aquaculture. In particular, optimum distribution and sufficient supply with EAAs is decisive. Since feed from natural protein sources such as soya, maize and wheat is generally deficient in certain EAAs, the targeted supplementing with synthetic EAAs, for example DL-methionine, L-lysine, L-threonine or L-tryptophan, on the one hand permits faster growth of the animals or increased milk production in high-yielding dairy cows, and on the other hand also more efficient utilization of the total feed. This represents a considerable economic advantage. The markets for feed additives are of great industrial and economic importance. Moreover, they are strong growth markets, which can be attributed in particular to the increasing importance of countries such as China and India.

L-methionine ((S)-2-amino-4-methylthiobutyric acid) represents, for many animal species, the first limiting amino acid of all EAAs and therefore has one of the most important roles in animal nutrition and as feed additive (Rosenberg et al., J. Agr. Food Chem. 1957, 5, 694-700). In the classical chemical synthesis, however, methionine is produced as a racemate, a 50:50 mixture of D- and L-methionine. This racemic DL-methionine can nevertheless be used directly as feed additive, because in some animal species under in vivo conditions there is a conversion mechanism, which transforms the unnatural D-enantiomer of methionine to the natural L-enantiomer. In this, the D-methionine is first deaminated by means of a nonspecific D-oxidase to α-keto-methionine and then further transformed with an L-transaminase to L-methionine (Baker, D. H. in “Amino acids in farm animal nutrition”, D'Mello, J. P. F. (ed.), Wallingford (UK), CAB International, 1994, 37-61). As a result, the available amount of L-methionine in the body is increased, and it can then be available to the animal for growth. The enzymatic conversion of D- to L-methionine has been observed in chicken, pigs and cows, but in particular also in fishes, shrimps and prawns. For example, Sveier et al. (Aquacult. Nutr. 2001, 7 (3), 169-181) and Kim et al. (Aquaculture 1992, 101 (1-2), 95-103) showed that the conversion of D- to L-methionine is possible in carnivorous Atlantic salmon and rainbow trout. The same was shown by Robinson et al. (J. Nutr. 1978, 108 (12), 1932-1936) and Schwarz et al. (Aquaculture 1998, 161, 121-129) for omnivorous fish species, for example catfish and carp. Furthermore, Forster and Dominy (J. World Aquacult. Soc. 2006, 37 (4), 474-480) showed, in feeding tests on omnivorous shrimps of the species Litopenaeus vannamei, that DL-methionine possesses the same efficacy as L-methionine. In 2007, globally more than 700,000 t of crystalline DL-methionine or racemic, liquid methionine hydroxy analog (MHA, rac-2-hydroxy-4-(methylthio)butanoic acid (HMB)) and solid calcium-MHA was produced and successfully used directly as feed additive for monogastric animals, e.g. poultry and pigs.

In contrast to methionine, with lysine, threonine and tryptophan in each case only the L-enantiomers can be used as feed additives, as the respective D-enantiomers of these three essential and limiting amino acids cannot be converted by the organism to the corresponding L-enantiomers in physiological conditions. Thus, the world market for L-lysine alone, the primary limiting amino acid for example in pigs, was over a million tonnes for the year 2007. For the other two limiting essential amino acids L-threonine and L-tryptophan, the world market in 2007 was over 100,000 t and a few 1000 t, respectively.

In monogastric animals, e.g. poultry and pigs, usually DL-methionine, MHA, as well as L-lysine, L-threonine and L-tryptophan are used directly as feed additive. In contrast, supplementing of feed with EAAs such as methionine, lysine, threonine or also MHA in ruminants is not effective, as most is degraded by microbes in the rumen of the ruminants. Owing to this degradation, therefore, only a fraction of the supplemented EAAs enters the animal's small intestine, where absorption into the blood generally takes place. Among the EAAs, mainly methionine has a decisive role in ruminants, as optimum supply is essential for high milk yield. For methionine to be available at high efficiency in ruminants, a rumen-resistant protected form must be used. There are several possible ways of endowing DL-methionine or rac-MHA with these properties. One possibility is to achieve high rumen resistance by applying a suitable protective layer or by distributing the methionine in a protective matrix. As a result, methionine can pass through the rumen practically without loss. Thereafter, the protective layer is then removed e.g. in the abomasum by acid hydrolysis and the methionine that is liberated can then be absorbed in the small intestine of the ruminant. Commercially available products are e.g. Mepron® from Evonik Degussa and Smartamine™ from Adisseo. The production and/or coating of methionine generally represents a technically complicated process and is therefore expensive. Moreover, the surface coating of the finished pellets can easily be damaged by mechanical stresses and abrasion during feed processing, which can lead to reduction or even complete loss of protection. Therefore, it is also not possible to process the protected methionine pellets to a larger mixed feed pellet, as once again this would disrupt the protecting layer through mechanical stress. This limits the use of these products. Another possibility for increasing rumen resistance is chemical derivatization of methionine or MHA. In this, the functional groups of the molecule are derivatized with suitable protecting groups. This can be achieved for example by esterification of the carboxylic acid function with alcohols. As a result, degradation in the rumen by microorganisms can be reduced. A commercially available product with chemical protection is, for example, Metasmart™, the racemic iso-propyl ester of MHA (HMBi). A biological value of at least 50% for HMBi in ruminants was disclosed in WO00/28835. Chemical derivatization of methionine or MHA often has the drawbacks of poorer bioavailability and comparatively low content of active substance.

In addition to the problems of degradation of supplemented EAA's such as methionine, lysine or threonine in the rumen in ruminants, various problems can also arise in fish and crustaceans when supplementing feed with EAAs. Through the rapid economic development of the farming of fish and crustaceans in highly industrialized aquaculture, optimum, economic and efficient means of supplementing essential and limiting amino acids have become more and more important in this area in recent years (Food and Agriculture Organization of the United Nations (FAO) Fisheries Department “State of World Aquaculture 2006”, 2006, Rome. International Food Policy Research Institute (IFPRI) “Fish 2020: Supply and Demand in Changing Markets”, 2003, Washington, D.C.). In contrast to chicken and pigs, use of crystalline EAAs as feed additive can lead to various problems with certain species of fishes and crustaceans. Thus, Rumsey and Ketola (J. Fish. Res. Bd. Can. 1975, 32, 422-426) report that the use of soya flour in combination with individually supplemented, crystalline amino acids did not lead to increased growth of rainbow trout. Murai et al. (Bull. Japan. Soc. Sci. Fish. 1984, 50 (11), 1957) were able to show that the daily feeding of fish diets with high dosages of supplemented, crystalline amino acids led in carp to more than 40% of the free amino acids being excreted via the gills and kidneys. Owing to the rapid absorption of supplemented amino acids shortly after food intake, there is a very rapid rise in amino acid concentration in the blood plasma of the fish (fast-response). At this time, however, the other amino acids from the natural protein sources, e.g. soya flour, are not yet in the plasma, which can lead to asynchronicity of the simultaneous availability of all important amino acids. A proportion of the highly concentrated amino acids is in consequence quickly excreted or quickly metabolized in the organism and utilized e.g. as a pure energy source. Because of this, in the carp there is little or no increase in growth when crystalline amino acids are used as feed additives (Aoe et al., Bull. Jap. Soc. Sci. Fish. 1970, 36, 407-413). In crustaceans, supplementing crystalline EAAs can also lead to other problems. Owing to the slow eating behavior of certain crustaceans, e.g. shrimps of the species Litopenaeus vannamei, because the feed is under water for a long time, leaching of the supplemented, water-soluble EAAs occurs, leading to the eutrophication of the body of water, instead of increased growth of the animals (Alam et al., Aquaculture 2005, 248, 13-16). Effective supply of fishes and crustaceans kept in aquaculture therefore requires, for certain species and applications, a special product form of the EAAs, for example, a suitably chemically or physically protected form. The aim is that, on the one hand, the product should remain sufficiently stable during feeding in the aqueous environment and should not be leached out of the feed. On the other hand, it should be possible for the amino acid product finally taken in by the animal to be utilized optimally and with high efficiency in the animal organism.

In the past, there have been many attempts to develop suitable feed additives, especially on the basis of the essential amino acids methionine and lysine, for fish and crustaceans. For example, WO8906497 describes the use of di- and tripeptides as feed additive for fish and crustaceans. This is said to promote growth of the animals. However, preferably di- and tripeptides from nonessential as well as nonlimiting amino acids, e.g. glycine, alanine and serine, were used, and these are present in more than sufficient amounts in many vegetable protein sources. Only DL-alanyl-DL-methionine and DL-methionyl-DL-glycine were described as methionine-containing dipeptides. Accordingly, the dipeptide only contains effectively 50% of active substance (mol/mol), which from the economic standpoint is to be regarded as very disadvantageous. WO02088667 describes the enantioselective synthesis and use of oligomers from MHA and amino acids, e.g. methionine, as feed additives, for fish and crustaceans, among others. It is said that faster growth can be achieved as a result. The oligomers described are synthesized by an enzyme-catalyzed reaction and have a very wide distribution of chain length of the individual oligomers. As a consequence the method is unselective, expensive and complicated in execution and purification.

Dabrowski et al. describe, in US20030099689, the use of synthetic peptides as growth-promoting feed additives for aquatic animals. In this case the peptides can represent a proportion by weight of 6-50% of the total feed formulation. The synthetic peptides preferably consist of EAAs. The enantioselective synthesis of these synthetic oligo- and polypeptides is, however, very complicated, expensive and difficult to scale up. Moreover, the efficacy of polypeptides of one individual amino acid is disputed, as often these are only converted very slowly, or not at all, to free amino acids in physiological conditions. For example, Baker et al. (J. Nutr. 1982, 112, 1130-1132) describe that because of its absolute insolubility in water, poly-L-methionine has no biological value for chicken, as absorption by the organism is not possible.

Diketopiperazines can be synthesized in several different ways. For example Jainta et al. (Eur. J. Org. Chem. 2008, 5418-5424) describe the microwave-assisted synthesis of cyclic dipeptides by condensation of amino acids. Zheng-Zheng et al. (Angew. Chem. Int. Ed., 2008, 47, 1758-1761) describe synthesis by means of biomimetic catalysis. In both cases solvents and/or catalysts are used, making cost-effective production of the cyclic dipeptides impossible.

Naraoka et al. (J. Chem. Soc. Perkin Trans. I, 1986, 1557-1560) converted amino acid esters, but the reaction rate was very slow in the selected reaction conditions and even after several days reaction had not gone to completion. Cyclic dipeptides can also be obtained from ordinary dipeptides by splitting off water. This was shown for example by Kopple et al. (J. Org. Chem., 1968, 33, 862-864) and Tullberg et al. (Tetrahedron, 2006, 62, 7484-7491). In both cases, however, flammable or toxic organic solvents have to be used. Snyder et al. obtained cyclic dipeptides by refunctionalization of existing diketopiperazine derivatives, e.g. chlorinated diketopiperazines (Journal of the American Chemical Society, 1944, 66, 1002-1004) or vinylated diketopiperazine derivatives (Journal of the American Chemical Society, 1944, 66, 511-512). Furthermore, Snyder et al. succeeded in synthesizing cyclic dipeptides from aminolactones (Journal of the American Chemical Society, 1942, 64, 2082-2084). In all these cases, educts involving complicated synthesis are required beforehand.

Another common method of synthesis of mixed cyclic dipeptides is the use of protecting group techniques, as employed e.g. by DesMarteau et al. (Tetrahedron Letters, 2006, 47, 561-564) or Egusa et al. (Bull Chem. Soc. Jpn., 1986, 59, 2195-2201). However, protecting group chemistry always requires additional reaction steps—on the one hand for protecting the amino or carboxylate group of the amino acids that are to be coupled, and on the other hand for removing the protecting groups again after coupling. A simplification is provided by solid phase synthesis. For example, Lloyd-Williams et al. (Pept. 1990, Proc. Eur. Pept. Symp. 21st, 1991, 146-148), Compo et al. (Tetrahedron, 2009, 65, 5343-5349) or Wang et al. (Tetrahedron Letters, 2002, 43, 865-867) produced cyclic dipeptides by means of solid phase chemistry. Solid phase synthesis is not suitable for use in the production of cyclic dipeptides at the kilogram scale, as the resins are excessively expensive.

In addition to the use of novel chemical derivatives of EAAs, e.g. methionine-containing peptides and oligomers, various possibilities for physical protection were also investigated, for example coatings or embedding an EAA in a protective matrix. For example, Alam et al. (Aquacult. Nutr. 2004, 10, 309-316 and Aquaculture 2005, 248, 13-19) showed that coated methionine and lysine, in contrast to uncoated products, have a very positive influence on the growth of young kuruma shrimps. Although the use of a special coating prevented leaching of methionine and lysine from the feed pellet, there are some serious disadvantages. The production and/or coating of amino acids is generally a technically complicated and challenging process and is therefore expensive. In addition, the surface coating of the finished coated amino acid can easily be damaged by mechanical stresses and abrasion during feed processing, which can lead to a decrease or even complete loss of physical protection. Furthermore, coating or the use of a matrix substance reduces the content of amino acid and is therefore often uneconomic.

### BRIEF DESCRIPTION OF THE DRAWINGS

FIG. 1 shows the solubility of cyclo-DL-Met-DL-Met in bile/water mixtures (bile obtained from mirror carp).

FIG. 2 shows the solubility of cyclo-DL-Met-DL-Met as a function of solution pH.

FIG. 3 shows the cleavage of DD/LL/meso-cyclo-Met-Met with enzymes from the rainbow trout.

FIG. 4 shows the cleavage of DD/LL-cyclo-Met-Met with enzymes from mirror carp.

FIG. 5 shows the cleavage of cyclo-L-His-L-His with enzymes from mirror carp.

FIG. 6 shows the cleavage of cyclo-D-Met-L-Leu with enzymes from mirror carp.

FIG. 7 shows the cleavage of cyclo-D-Met-L-Phe with enzymes from rainbow trout.

FIG. 8 shows the cleavage of cyclo-D-Met-L-Lys with enzymes from rainbow trout.

FIG. 9 shows the cleavage of cyclo-D-Met-L-Thr with enzymes from whiteleg shrimp.

### DETAILED DESCRIPTION OF THE PREFERRED EMBODIMENTS

Against the background of the disadvantages of the conventional methods, the main object of the invention was to provide a chemically protected product by a covalently bound combination of two essential and limiting amino acids e.g. DL-methionine, L-lysine, L-threonine or L-tryptophan for ruminants, for example dairy cows, but also for many omnivorous, herbivorous and carnivorous fish and crustacean species that live in salt water or fresh water. In particular, this chemically protected product may possess a “slow release” mechanism, i.e. slow, continuous release of free methionine and EAA (EAA=essential amino acid) in physiological conditions. Moreover, the chemically protected product form prepared from two identical or different EAAs may be rumen-resistant and so may be suitable for all ruminants. For use as feed additive for fish and crustaceans, the product form may display low solubility from the total feed pellet or extrudate in water (leaching).

Furthermore, the feed additive may have better solubility in the digestive system of fishes and crustaceans than in the surrounding salt water or fresh water.

Another object was to identify a substitute for crystalline EAAs as feed or a feed additive with very high biological value, which may have good handling, storage and stability properties in the usual conditions of mixed feed processing, in particular pelletization and extrusion.

In this way, for ruminants, fish and crustaceans, in addition to the known crystalline, coated or matrix protected EAAs, additional efficient sources of essential amino acids may be made available, which as far as possible do not have the disadvantages of the known products, or have said disadvantages to a reduced extent.

The present invention provides a feed or a feed additive for animal nutrition based on a six-membered heterocyclic ring system (2,5-piperazinedione, diketopiperazine [DKP], cyclo-dipeptide, dehydrodipeptide), wherein amino acid residues of essential and limiting amino acids, e.g. DL-methionine, L-lysine, L-threonine and L-tryptophan, are bound covalently in the 3,6-positions of the diketopiperazine, and which may be used as feed additive for the feeding of ruminants, e.g. dairy cows, in particular but also of fishes and crustaceans in aquaculture.

The object is achieved with a feed additive containing at least one diketopiperazine (cyclic dipeptide) with the following general formula IV or a salt thereof:

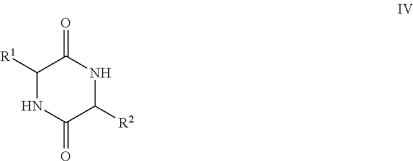

where R¹ and R² independently of one another represent an amino acid residue R (preferably in the L-configuration) selected from the group comprising methionine (R=—(CH₂)₂SCH₃), lysine (R=—(CH₂)₄NH₂), threonine (R=—CH(OH)(CH₃)), tryptophan (R=-indolyl), histidine (R=-imidazoyl), valine (R=—CH(CH₃)₂), leucine (R=—CH₂CH((CH₃)₂), isoleucine (R=—CH(CH₃)CH₂CH₃), phenylalanine (R=—CH₂Ph), arginine (R=—(CH₂)₃NHC(═NH)NH₂), cysteine (R=—CH₂SH), where optionally R¹ can be the same as R²;
or containing at least one compound with the following general formula V or a salt thereof, where R¹ and R² are as defined above

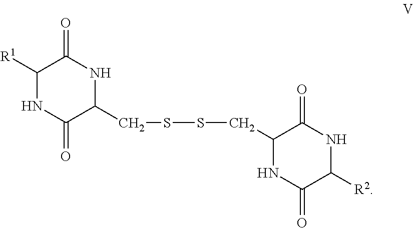

In a preferred embodiment R¹ and/or R² are in the L-configuration.

In a preferred embodiment of the feed additive R¹ or R² is a methionyl residue (R=—(CH₂)₂SCH₃) in the DD-, LL-, LD- or DL-configuration.

Furthermore, it is preferable for the diketopiperazine contained in the feed additive to be in the form of cyclo-D-EAA-D-EAA, cyclo-L-EAA-D-EAA, cyclo-D-EAA-L-EAA, cyclo-L-EAA-L-EAA or mixtures thereof, in particular as a diastereomeric mixture cyclo-DL-EAA-DL-EAA, where EAA denotes an amino acid selected from the group comprising methionine, lysine, threonine, tryptophan, histidine, valine, leucine, isoleucine, phenylalanine, arginine, cysteine and cystine.

Moreover, it is further preferred that R¹ and R² in each case represent a methionyl residue (R=—(CH₂)₂SCH₃), and with the diketopiperazine in the DD-, LL-, DL- or LD-configuration or in mixtures thereof; it is preferable (i.e. when R¹ and R² equal —(CH₂)₂SCH₃) if the diketopiperazine with the LL-configuration is only present in a mixture with other configurations.

It is especially preferable for the diketopiperazine contained in the feed additive to be in the form of a diastereomeric mixture DD/LL/meso-cyclo-Met-Met (i.e. as a mixture of DD/LL-cyclo-Met-Met and meso-cyclo-Met-Met), where Met denotes methionine.

The invention further relates to a feed mixture containing the feed additive described above.

In a preferred embodiment of the feed mixture, the mixture additionally contains: one or more of the following substances: DL-methionine, L-EAA, DL-EAA, the diastereomeric mixtures DD/LL/DL/LD-methionyl-EAA and/or DD/LL/DL/LD-EAA-methionine, DD/LL-methionyl-EAA, DD/LL-EAA-methionine, D-methionyl-L-EAA, L-methionyl-L-EAA, D-methionyl-D-EAA, L-methionyl-D-EAA, D-EAA-L-methionine, L-EAA-L-methionine, D-EAA-D-methionine, L-EAA-D-methionine, preferably in each case additionally mixed with DL-methionine, preferably with a proportion of DL-methionine from 0.01 to 90 wt. %, preferably from 0.1 to 50 wt. %, especially preferably from 1 to 30 wt. %, preferably in each case additionally mixed with an L-EAA, for example L-lysine, preferably with a proportion of L-EAA from 0.01 to 90 wt. %, preferably from 0.1 to 50 wt. %, especially preferably from 1 to 30 wt. %.

The feed additive containing diketopiperazines (cyclic dipeptides) and salts thereof may be suitable as an additive in feed mixtures for ruminants, in particular but also for fish and crustaceans in aquaculture. Use as additive in feed mixtures for ruminants may be especially preferred.

The feed mixture preferably contains 0.01 to 5.0 wt. %, more preferably 0.05 to 0.5 wt. % of diketopiperazine, alone or mixed with one or more free amino acids (EAA), mixed with one or more natural or unnatural dipeptides (EAA-EAA) or in a mixture containing amino acids (EAA) and dipeptides (EAA-EAA).

The use of 3,6-bis[2-(methylthio)ethyl]-2,5-piperazinedione (cyclo-Met-Met, methionine-diketopiperazine) may be especially advantageous, because this diketopiperazine displays particularly good leaching behavior, based on its low solubility.

Furthermore, the compound displays good pelletizing and extrusion stability in feed production. The diketopiperazines may be stable in mixtures with the usual components and feeds, e.g. cereals (e.g. maize, wheat, triticale, barley, millet, etc.), vegetable or animal protein carriers (e.g. soybeans and rape and products from further processing thereof, legumes (e.g. peas, beans, lupines, etc.), fish-meal, etc.) and in combination with supplemented essential amino acids, proteins, peptides, carbohydrates, vitamins, minerals, fats and oils.

A further advantage may be that because of the particularly high proportion of active substance of diketopiperazines per kg of substance, compared with two mol of amino acids per mol of diketopiperazine, there is a saving of two moles of water.

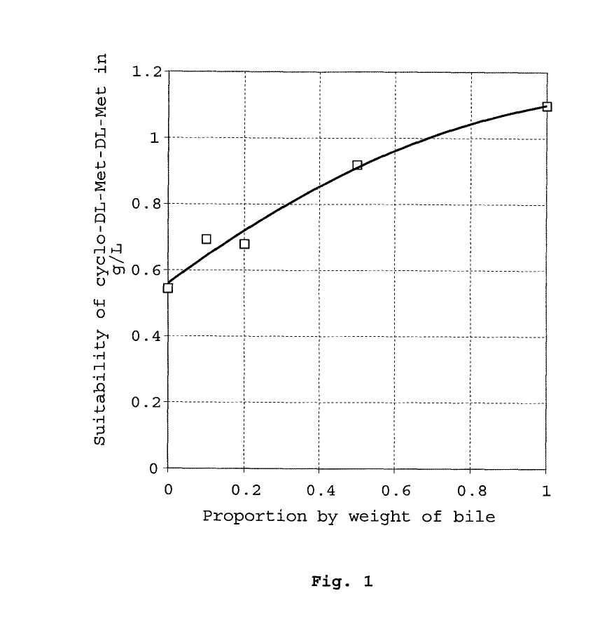

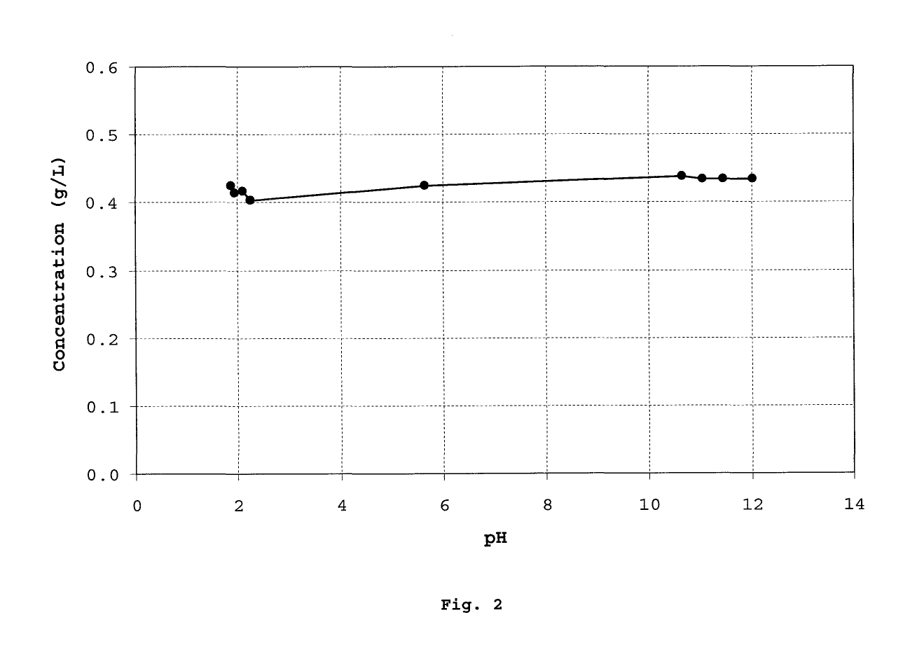

Furthermore, the diketopiperazine may be especially suitable as feed additive for fish and crustaceans raised in aquaculture, as the solubility of the diketopiperazine may generally be very low (see FIG. 2), but may be more soluble in the digestive tract of fishes or crustaceans than in the surrounding water (see FIG. 1).

In a preferred use the feed mixture contains proteins and carbohydrates, preferably based on fish, soya or corn flour, and may be supplemented with essential amino acids, proteins, peptides, vitamins, minerals, carbohydrates, fats and oils.

In particular it may be preferable for the feed mixture to contain cyclo-EAA-EAA alone as cyclo-L-EAA-L-EAA, cyclo-D-EAA-L-EAA, cyclo-L-EAA-D-EAA, cyclo-D-EAA-D-EAA or mixed with one another, in particular as a diastereomeric mixture of cyclo-DL-EAA-DL-EAA, preferably in each case additionally mixed with L-EAA, D-EAA or DL-EAA, for example methionine, lysine, threonine or tryptophan, in each case alone or mixed with one another, preferably with a proportion of amino acid from 0.01 to 90 wt. %, preferably from 0.1 to 50 wt. %, especially preferably from 1 to 30 wt. %.

Furthermore, it may be preferable for the feed mixture to contain cyclo-EAA-EAA alone as cyclo-L-EAA-L-EAA, cyclo-D-EAA-L-EAA, cyclo-L-EAA-D-EAA, cyclo-D-EAA-D-EAA or mixed with one another, in particular as a diastereomeric mixture cyclo-DL-EAA-DL-EAA, preferably in each case additionally mixed with dipeptides of the general formula EAA-EAA, alone as L-EAA-L-EAA, D-EAA-L-EAA, L-EAA-D-EAA and D-EAA-D-EAA or mixed with one another, in particular as a diastereomeric mixture DL-EAA-DL-EAA, preferably in each case additionally mixed with L-EAA, D-EAA or DL-EAA, for example methionine, lysine, threonine or tryptophan, in each case alone or mixed with one another, preferably with a proportion of amino acid and/or proportion of dipeptide from 0.01 to 90 wt. %, preferably from 0.1 to 50 wt. %, especially preferably from 1 to 30 wt. %.

According to the invention, the diketopiperazine may be cyclo-EAA-EAA alone as cyclo-L-EAA-L-EAA, cyclo-D-EAA-L-EAA, cyclo-L-EAA-D-EAA, cyclo-D-EAA-D-EAA or mixed with one another, in particular as a diastereomeric mixture of cyclo-DL-EAA-DL-EAA, or in the case of charged EAA residues e.g. in the case of lysine, histidine or arginine the alkali and alkaline-earth salts thereof, for example as the sparingly soluble calcium or zinc salts, alone or mixed with in each case additionally mixed with dipeptides of the general formula EAA-EAA, alone as L-EAA-L-EAA, D-EAA-L-EAA, L-EAA-D-EAA and D-EAA-D-EAA or mixed with one another, in particular as a diastereomeric mixture DL-EAA-DL-EAA, preferably in each case additionally mixed with L-EAA, D-EAA or DL-EAA, preferably used for ruminants and especially preferably for fish and crustaceans (see Scheme 1):

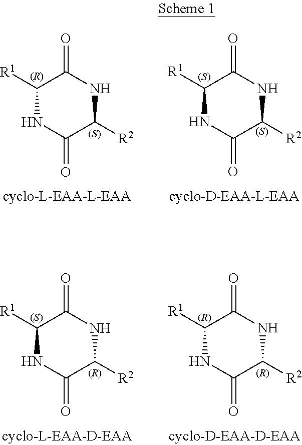

The residues R¹ and R² of the EAAs represent:
R=2-(methylthio)ethyl)-(methionine)
R=1-methylethyl-(valine)
R=2-methylpropyl-(leucine)
R=(1S)-1-methylpropyl-(isoleucine)
R=(1R)-1-hydroxyethyl-(threonine)
R=4-aminobutyl-(lysine)
R=3-[(aminoiminomethyl)-amino]propyl-(arginine)
R=benzyl-(phenylalanine)
R=(1H-imidazol-4-yl)methyl-(histidine)
R=(1H-indol-3-yl)methyl-(tryptophan)
R=mercaptomethyl-(cysteine)
In the case of cystine, there is a compound of formula (cyclo-EAA-cystine)-S—S-(cyclo-Cys-EAA).

In a preferred use, the animals raised in aquaculture may be fresh and salt water fishes and crustaceans selected from the group comprising carp, trout, salmon, catfish, perch, flatfish, sturgeon, tuna, eels, bream, cod, shrimps, hill and prawns, quite especially silver carp (Hypophthalmichthys molitrix), grass carp (Ctenopharyngodon idella), scaly carp (Cyprinus carpio) and bighead carp (Aristichthys nobilis), crucian carp (Carassius carassius), catla (Catla catla), rohu (Labeo rohita), Pacific and Atlantic salmon (Salmo salar and Oncorhynchus kisutch), rainbow trout (Oncorhynchus mykiss), American catfish (Ictalurus punctatus), African catfish (Clarias gariepinus), pangasius (Pangasius bocourti and Pangasius hypothalamus), Nile tilapia (Oreochromis niloticus), milkfish (Chanos chanos), cobia (Rachycentron canadum), whiteleg shrimp (Litopenaeus vannamei), black tiger shrimp (Penaeus monodon) and giant river prawn (Macrobrachium rosenbergii).

The main subject of the present invention may be the use of diketopiperazines (cyclo-dipeptides) alone as cyclo-L-EAA-L-EAA, cyclo-D-EAA-L-EAA, cyclo-L-EAA-D-EAA, cyclo-D-EAA-D-EAA or mixed with one another, in particular as a diastereomeric mixture cyclo-DL-EAA-DL-EAA, as growth promoter for ruminants, but also for omnivorous, carnivorous and herbivorous fishes and crustaceans in aquaculture. Moreover, by using cyclo-DL-EAA-DL-EAA as feed additive, milk production may be increased in high-yielding dairy cows.

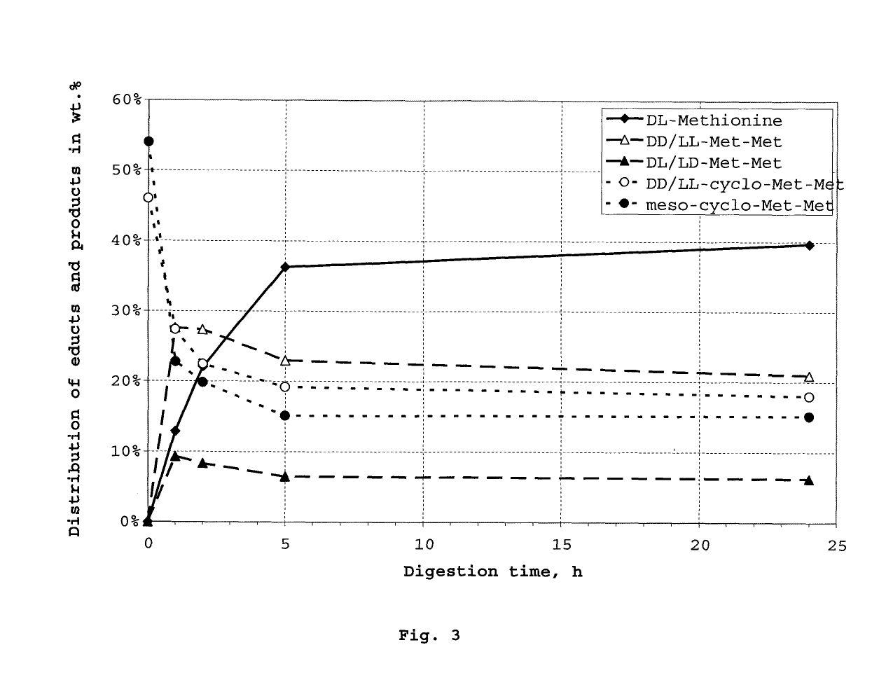

Thus, according to the invention, DD/LL/meso-cyclo-Met-Met as a diastereomeric mixture of a 50:50 mixture of DD/LL-cyclo-Met-Met and meso-cyclo-Met-Met, may be cleaved enzymatically in physiological conditions by fish, e.g. carp and trout, to free D- or L-methionine (see FIG. 3).

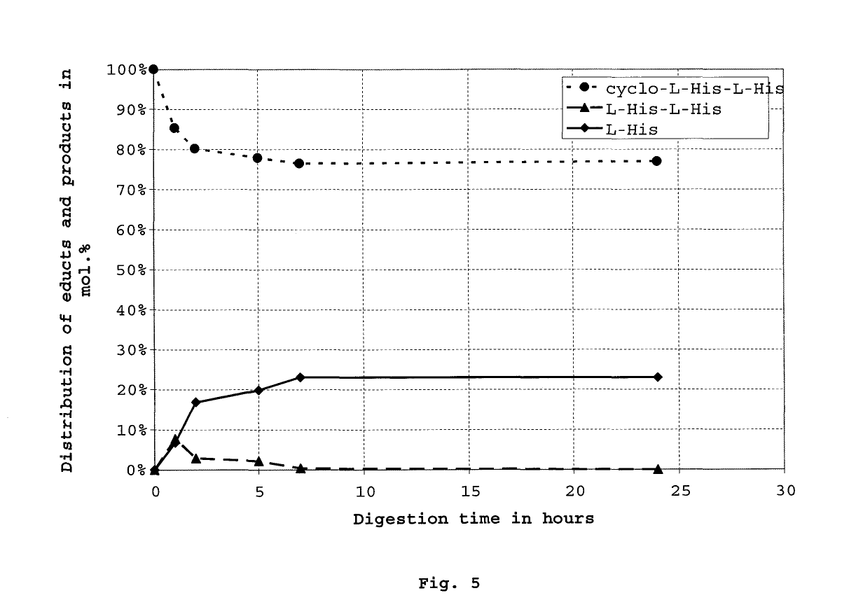

Also shown, according to the invention, mixed cyclic dipeptides, e.g. cyclo-D-Met-L-Leu, cyclo-D-Met-L-Phe or cyclo-D-Met-L-Lys may be cleaved enzymatically in physiological conditions by digestive enzymes from mirror carp in in vitro cleavage tests (see FIGS. 5-8). Therefore unnatural cyclic dipeptides with D-amino acids (D-EAA) may also be suitable as feed additives (see Scheme 2).

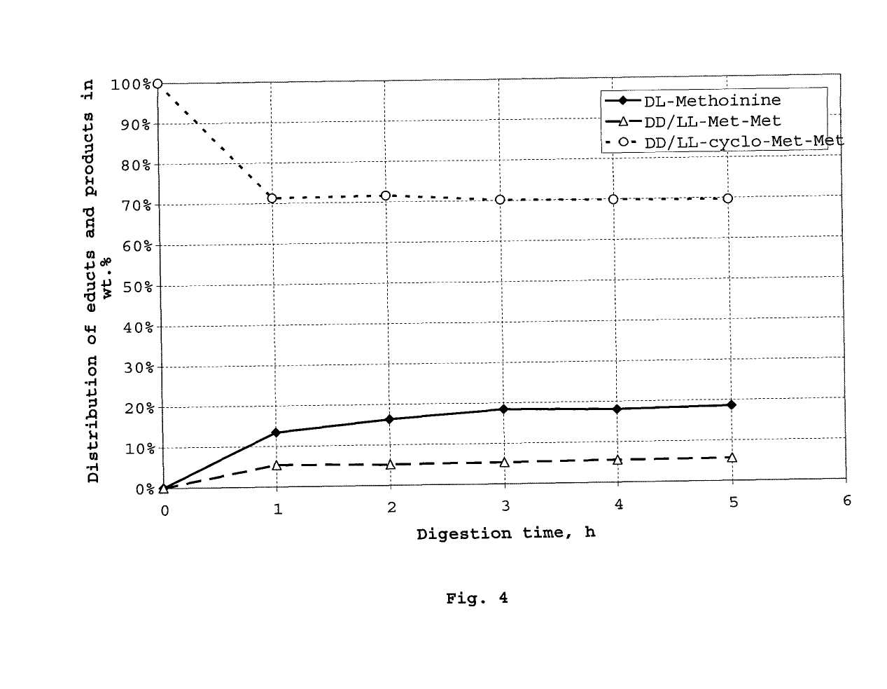

Digestive enzymes were isolated from omnivorous carp and carnivorous trout and were reacted in optimized in vitro experiments under physiologically comparable conditions with DD/LL/meso-cyclo-Met-Met as diastereomeric mixture of a 50:50 mixture of DD/LL-cyclo-Met-Met and meso-cyclo-Met-Met. The characteristic feature of the cleavage of DD/LL/meso-cyclo-Met-Met according to the invention may be that in addition to the diastereomer cyclo-L-Met-L-Met occurring naturally in food, the diastereomers cyclo-D-Met-L-Met and cyclo-D-Met-D-Met may also be cleaved in physiological conditions (see FIGS. 3 and 4). In in vitro conditions, isolated enzyme cocktails from digestive systems of fishes are only active for a short time, so that over a reaction time of several hours the cleavage rate decreases dramatically and finally comes to a standstill, although the cyclic dipeptide has not yet been converted completely. It may be assumed that in in vivo conditions in live fish, the enzyme activity may be much higher, remains stable through constant replenishment of the enzymes and finally leads to complete utilization of the feed additive.

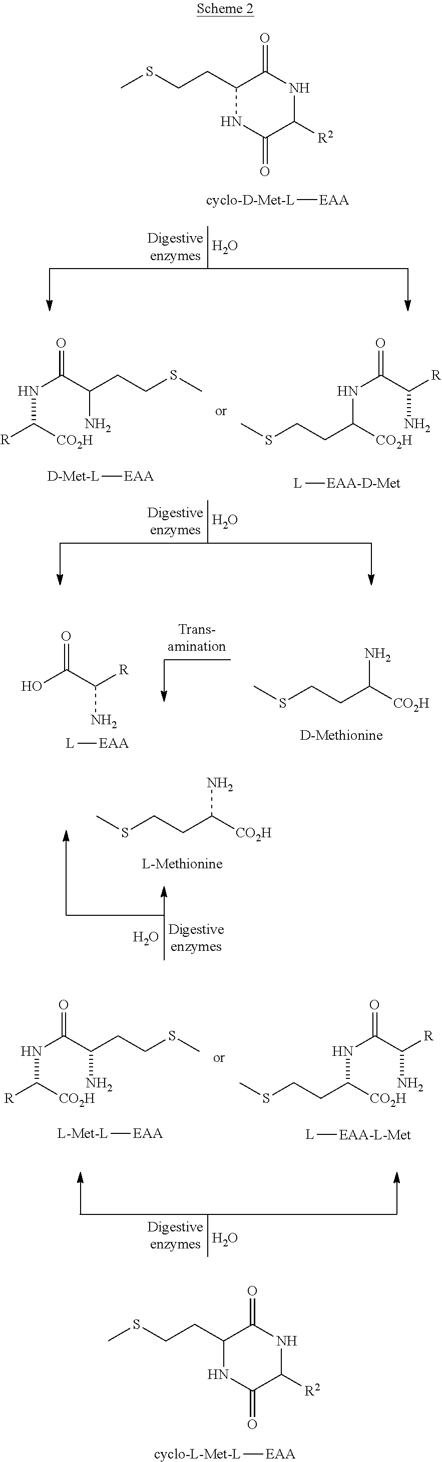

The diketopiperazines were digested in vitro with digestive enzymes from carnivorous rainbow trout and omnivorous mirror carp. For this, the enzymes were removed from the digestive tracts of fishes and shrimps. The enzyme solutions obtained were then added to the diketopiperazines. For better comparability of the digestibility of dipeptides of various species, identical conditions were selected for the in vitro digestion studies (37° C., pH 9).

As can be seen from FIGS. 3 and 4, the diastereomeric mixture DD/LL/meso-cyclo-Met-Met or the diastereomer DD/LL-cyclo-Met-Met can be cleaved by digestive enzymes of the rainbow trout and of the mirror carp. In these examples, enzymatic cleavage was not quantitative, because in the in vitro digestion experiments the enzymes are only stable for a short time in the chosen conditions (37° C., pH 9) and the enzyme activity decreases dramatically after just a short time. It may be assumed that the reaction is much faster and more efficient in in vivo conditions.

It follows from the results obtained that both natural cyclic dipeptides (e.g. cyclo-L-Met-L-Met), and unnatural cyclic dipeptide (e.g. cyclo-D-Met-D-Met and cyclo-D-Met-L-Met) can be cleaved by digestive enzymes of carnivorous and omnivorous fish species in vitro. By adding natural and unnatural cyclo-Met-Met diketopiperazines to the feed, deficient essential amino acids (here: DL-Met) may thus be dosed specially.

The object of the invention may in addition be achieved with a diketopiperazine or a salt thereof, with the following general formula IV:

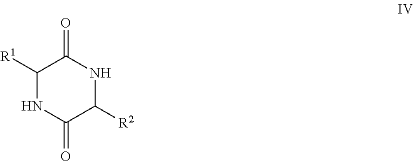

where R¹ and R² independently of one another represent an amino acid residue selected from the group comprising methionine (R^{1/2}=—(CH₂)₂SCH₃), lysine (R^{1/2}=—(CH₂)₄NH₂), threonine (R^{1/2}=—CH(OH)(CH₃)), tryptophan (R^{1/2}=-indolyl), histidine (R^{1/2}=-imidazoyl), valine (R^{1/2}=—CH(CH₃)₂), leucine (R^{1/2}=—CH₂CH((CH₃)₂), isoleucine (R^{1/2}=—CH(CH₃)CH₂CH₃), phenylalanine (R^{1/2}=—CH₂Ph), arginine (R^{1/2}=—(CH₂)₃NHC(═NH)NH₂), cysteine (R^{1/2}=—CH₂SH), where optionally R¹ can be the same as R²;
with the proviso that when R¹ and R² equal —(CH₂)₂SCH₃, the diketopiperazine is not exclusively in the form of cyclo-L-Met-L-Met;
or a compound with the following general formula V or a salt thereof, where R¹ and R² are as defined above

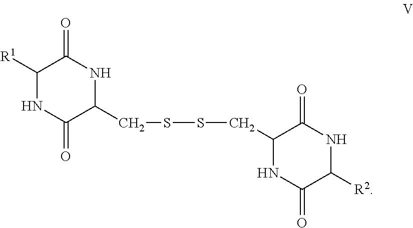

In a preferred embodiment the carbon atoms with R¹ and/or R² may be in the L-configuration.

In another preferred embodiment R¹ or R² may be a methionyl residue (R=—(CH₂)₂SCH₃) in the DD-, LL-, LD- or DL-configuration on the accompanying carbon atoms.

Preferably the diketopiperazine may be in the form of cyclo-D-EAA-D-EAA, cyclo-L-EAA-D-EAA, cyclo-D-EAA-L-EAA, cyclo-L-EAA-L-EAA or mixtures thereof, in particular as a diastereomeric mixture cyclo-DL-EAA-DL-EAA, where EAA denotes an amino acid selected from the group comprising methionine, lysine, threonine, tryptophan, histidine, valine, leucine, isoleucine, phenylalanine, arginine, cysteine and cystine.

It may be further preferred that R¹ and R² each represent a methionyl residue (R=—(CH₂)₂SCH₃), with the diketopiperazine in the DD-, LL-, DL- or LD-configuration or in mixtures thereof; with the proviso that if R¹ and R² equal —(CH₂)₂SCH₃, the diketopiperazine with the LL-configuration may only be present mixed with other configurations.

In another embodiment the diketopiperazine may be in a mixture as DD/LL/meso-cyclo-Met-Met, preferably in a 50:50 mixture of DD/LL-cyclo-Met-Met and meso-cyclo-Met-Met.

The present invention also provides a use of the diketopiperazines as feed additive for ruminants, fresh or salt water fishes and crustaceans.

The object of the invention is moreover achieved with a method of production of a diketopiperazine with the following general formula IV or a salt thereof:

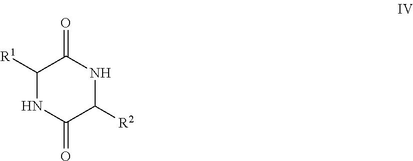

or a compound with the following general formula V or a salt thereof,

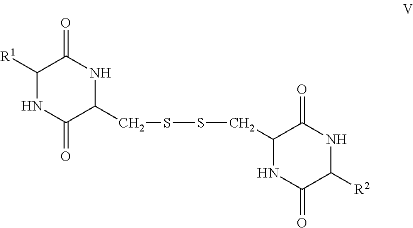

from one or more amino acid esters of the following general formula III:

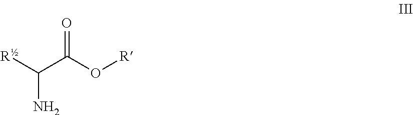

where R¹ and R² independently of one another are defined as follows:

### Formula IV and V:

R^{1/2}=2-(methylthio)ethyl)-(methionine)
R^{1/2}=1-methylethyl-(valine)
R^{1/2}=2-methylpropyl-(leucine)
R^{1/2}=(1S)-1-methylpropyl-(isoleucine)
R^{1/2}=(1R)-1-hydroxyethyl-(threonine)
R^{1/2}=4-aminobutyl-(lysine)
R^{1/2}=3-[(aminoiminomethyl)-amino]propyl-(arginine)
R^{1/2}=benzyl-(phenylalanine)
R^{1/2}=(1H-imidazol-4-yl)methyl-(histidine)
R^{1/2}=(1H-indol-3-yl)methyl-(tryptophan)
R^{1/2}=mercaptomethyl-(cysteine)

### Formula III:

R^{1/2}=—CH₂—S—S—CH₂—CH(NH₂)COOR′(cystine)
where optionally R¹ can be equal to R²;
and where R′ defines linear or branched aliphatic residues or aromatic residues and different R′ may occur in different amino acid ester molecules;
where reaction of the amino acid ester to the diketopiperazine takes place in substance.

In a preferred method the amino acid ester of general formula III may be obtained by esterification of an amino acid with the general formula I R^{1/2}—CH(NH₂)—COOH or cystine with a compound with the general formula II R′—OH.

Furthermore, it is preferable for the esterification to be carried out in the presence of a strong acid, preferably in the presence of HCl or H₂SO₄.

In a preferred method the residue R′ may be a C₁-C₈-alkyl residue, more preferably a C₁-C₆-alkyl residue and especially preferably a C₁-C₄-alkyl residue, where the alkyl residue may be linear or optionally, branched.

In a preferred method the residue R′ may be selected from the group comprising methyl, ethyl, n-propyl, iso-propyl, n-butyl, iso-butyl, sec-butyl, benzyl.

In another preferred method the residue R′ may be a C₂-C₈-alkenyl residue, more preferably a C₂-C₆-alkenyl residue and especially preferably a C₂-C₄-alkenyl residue, where the alkenyl residue may be linear or optionally branched.

In a preferred method the amino acid ester may be concentrated before the reaction of the amino acid ester to the diketopiperazine.

As mentioned above, reaction of the amino acid ester to the diketopiperazine according to the invention takes place in substance and in fact without the use of solvents. This means that according to the invention, during reaction of the amino acid ester to the diketopiperazine, no solvents, in particular no organic, polar or aqueous solvents, and in particular preferably no bases are present, except the following substances: the diketopiperazine itself, forming in the reaction, and the compound with the general formula R′—OH, which is removed by distillation during the reaction.

Especially preferably, reaction of the amino acid ester to the diketopiperazine takes place without the use of transamidation catalysts.

In an especially preferred embodiment, reaction of the amino acid ester to the diketopiperazine may take place in pure substance, preferably with a purity of >50 wt. %, preferably >90 wt. %, especially preferably >95 wt. % and quite especially preferably >98 wt. %.

In another preferred method, reaction of the amino acid ester to the diketopiperazine may be carried out at a temperature from 30 to 220° C., preferably at a temperature from 50 to 170° C. and especially preferably at a temperature from 70 to 140° C.

In the method according to the invention, conversion of the amino acid ester to the diketopiperazine may preferably be carried out by separating the compound with the general formula R′—OH (e.g. an alkanol) by distillation, for example under its own pressure, at normal pressure or at reduced pressure, preferably at a pressure from 0.01 to 20 bar, especially preferably at a pressure from 0.05 to 1.5 bar, quite especially preferably at atmospheric pressure. Preferably, in said distillation the diketopiperazine may be obtained by crystallization.

In another preferred method, the amino acid ester that was not converted completely in the reaction may be recovered and returned to the process.

Also in another preferred method, the compound with the general formula R′—OH not completely converted in the esterification of the amino acid to the amino acid ester and/or obtained again in the reaction of the amino acid ester to the diketopiperazine may be recovered and returned to the process.

In particular it may be preferable for the amino acid ester in the DL-, L- or D-configuration from the group methionine, lysine, threonine, tryptophan, histidine, valine, leucine, isoleucine, phenylalanine, arginine, cysteine, cystine to be heated in pure substance without the use of solvents. Both the compound R′—OH that is split off (e.g. an alkanol), and unreacted amino acid ester may be completely recycled and returned to the process (see Schemes 3 and 4). In the method according to the invention, the ester may be preferably obtained first from the free amino acid suspended in a compound R′—OH (e.g. an alkanol) with elimination of water by adding a strong acid, e.g. HCl or H₂SO₄. After release of the amino acid ester, e.g. from its hydrochloride, by means of a base e.g. NH₃ or K₂CO₃ and concentration, the resultant oil (i.e. the diketopiperazine in pure substance) may be heated, to separate the alcohol by distillation and crystallize the cyclic dipeptide (diketopiperazine of formula IV) highly selectively from the reaction mixture. After filtration and washing of the product with a solvent, unreacted amino acid ester may be recovered and may be returned to the process again.

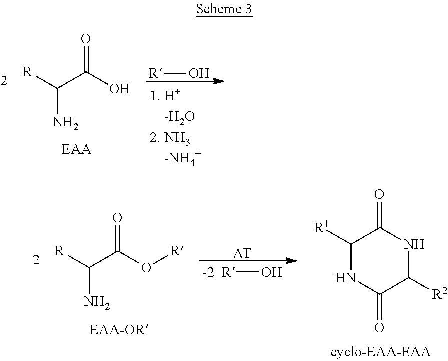

It should be noted that Scheme 3 is shown simplified, as the reaction scheme does not take into account that various amino acid molecules may be used or various amino acid esters may be used, relative to the amino acid residue but also possibly with respect to R′.

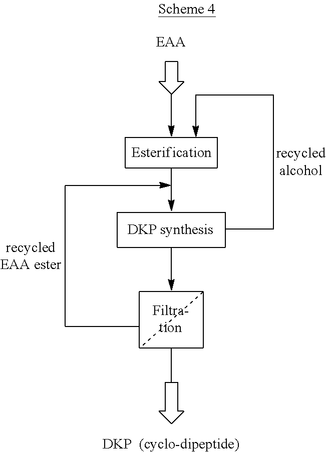

In another embodiment of the method according to the invention, for synthesis of mixed diketopiperazines of general formula cyclo-EAA-EAA, two or more different amino acid esters in the DL-, L- or D-configuration from the group comprising methionine, lysine, threonine, tryptophan, histidine, valine, leucine, isoleucine, phenylalanine, arginine, cysteine, cystine in any mixtures with one another, may be reacted.

Furthermore, the method of the present invention may be carried out in batch processes known by a person skilled in the art or in continuous processes.

Having generally described this invention, a further understanding can be obtained by reference to certain specific examples which are provided herein for purposes of illustration only, and are not intended to be limiting unless otherwise specified.

### EXAMPLES

### Example 1

### Synthesis of DL-methionine methyl ester (DL-Met-OMe)

200 g (1.34 mol) of DL-methionine was suspended in 1 L methanol. HCl gas was led into this suspension until the solid had dissolved and then was led in for a further 1 hour. The temperature rose to 60° C. Then approx. 200 mL methanol was distilled from the reaction solution at 30° C. in a rotary evaporator, and during this most of the excess HCl gas was removed. Then NH₃ gas was led into the remaining solution at 10-20° C. until the reaction solution had a definite alkaline reaction. The precipitated NH₄Cl was drawn off by suction and was washed with methanol. The filtrate was concentrated in the rotary evaporator, taken up in 750 mL ethyl acetate, and washed twice with 50 mL of 10% K₂CO₃ solution each time, dried over MgSO₄ and concentrated in the rotary evaporator.

Final weight: 203 g (93% of theoretical) of slightly yellowish clear oil.

¹H and ¹³C-NMR agreed with values in the literature.

### Example 2

### Synthesis of DL-methionine-iso-propyl ester (DL-Met-OiPr)

200 g (1.34 mol) of DL-methionine was suspended in 1 L iso-propanol. HCl gas was led into this suspension until the solid had dissolved and then was led in for a further 1 hour. The temperature rose to 60° C. Then approx. 200 mL iso-propanol was distilled from the reaction solution at 30° C. in the rotary evaporator, and during this most of the excess HCl gas was removed. NH₃ gas was then led into the remaining solution at 10-20° C. until the reaction solution had a definite alkaline reaction. The precipitated NH₄Cl was removed with suction and washed with iso-propanol. The filtrate was concentrated in the rotary evaporator, taken up in 750 mL of ethyl acetate, washed twice with 50 mL of 10% K₂CO₃ solution each time, dried over MgSO₄ and concentrated in the rotary evaporator.

Final weight: 233 g (91% of theoretical) of slightly yellowish clear oil.

¹H and ¹³C-NMR agreed with values in the literature.

### Example 3

### Synthesis of 3,6-bis[2-(methylthio)ethyl]-2,5-piperazinedione (DD/LL/meso-cyclo-Met-Met) from DL-methionine methyl ester (DL-Met-OMe)

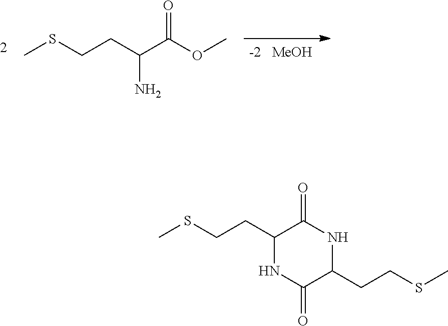

272 g (1.67 mol) of DL-methionine methyl ester was heated to 130° C., stirring well, and stirred at this temperature for 2 hours. 36 g methanol was distilled off and the cyclo-Met-Met crystallized out. After cooling, 250 mL methanol was added to the crystal slurry, it was stirred briefly, filtered with suction, washed with methanol and dried in the vacuum drying cabinet at 30° C. The filtrate was concentrated in the rotary evaporator at 40° C. and returned to the next batch.

Final weight: 149 g (68% of theoretical) of white solid, purity >99% (HPLC), melting point 235-236° C.

¹H-NMR of 3,6-bis[2-(methylthio)ethyl]-2,5-piperazinedione (500 MHz, d₆-DMSO): δ=1.85-2.05 (m, 4H, 2×SCH₂CH₂); 2.049 (s, 6H, 2×SCH₃); 2.46-2.60 (m, 4H, 2×SCH₂); 3.92-3.99 (m, 2H, 2×CH); 8.213 (s, 2H, 2×NH)

¹³C-NMR of 3,6-bis[2-(methylthio)ethyl]-2,5-piperazinedione (125.8 MHz, d₆-DMSO): δ=14.35 (CH₃); 14.38 (CH₃); 28.50 (CH₂S); 28.68 (CH₂S); 31.92 (CH₂CH₂S); 32.33 (CH₂CH₂S); 52.92 (CH); 52.96 (CH); 167.69 (C═O); 167.71 (C═O)

Elemental analysis for C₁₀H₁₈N₂O₂S₂ (M=262.39 g/mol):

Calculated: C, 45.77; H, 6.91; N, 10.68; S, 24.44

Found: C, 45.94; H, 6.96; N, 10.64; S, 24.38

### Example 4

### Synthesis of 3,6-bis-(S)-(1H-imidazol-4-yl)-2,5-piperazinedione (cyclo-L-His-L-His) from L-histidine methyl ester (L-His-OMe)

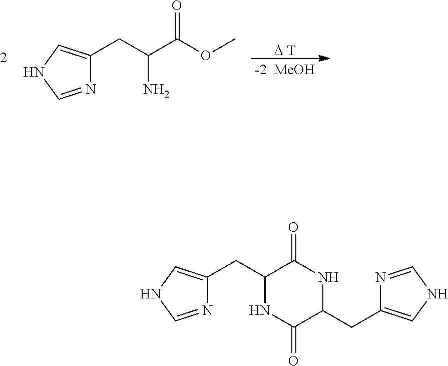

8.46 g (50 mmol) of L-histidine methyl ester (L-His-OMe) was heated to 80° C. while stirring well, and stirred at this temperature for 5 hours in a water jet vacuum. After cooling, 25 mL ethyl acetate was added to the crystal slurry, it was stirred briefly, filtered with suction, washed with ethyl acetate and dried in an oil-pump vacuum.

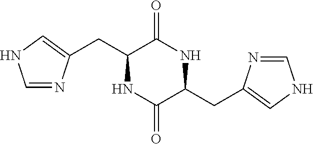

Final weight: 7.80 g (57% of theoretical) as white solid.

¹H-NMR of 3,6-bis-(S)-(1H-imidazol-4-yl)-2,5-piperazinedione (cyclo-L-His-L-His) (500 MHz, d₆-DMSO): δ=2.94 (dd, ³J=6.8 Hz, ¹J=15.4 Hz, 2H, 2×CH′H″); 3.07 (dd, ³J=3.8 Hz, ¹J=15.4 Hz, 2H, 2×CH′H″); 4.30-4.34 (m, 2H, 2×CH); 7.31 (bs, 2H, 2×N═CH—N); 8.17 (bs, 2H, 2×NH); 8.82 (bs, 2H, 2×NH—CH═C); 13-15 (bs, 2H, 2×CH—NH—CH)

### Example 5

### Synthesis of cyclic cyclo-Met-Leu dipeptides from DL-methionine methyl ester (DL-Met-OMe) and L-leucine methyl ester (L-Leu-OMe) and isolation of a diastereomer 3-(R)-[2-(methylthio)ethyl]-6-(S)-(2-(methyl)propyl)-2,5-piperazinedione (cyclo-D-Met-L-Leu)

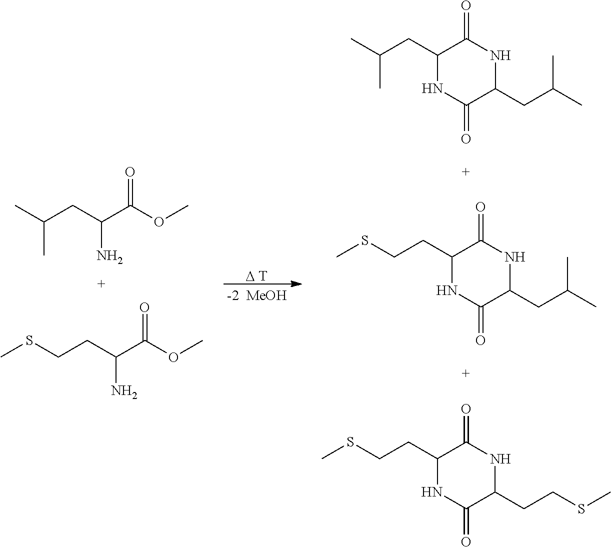

4.08 g (25 mmol) of DL-methionine methyl ester (DL-Met-OMe) and 3.63 g (25 mmol) of L-leucine methyl ester (L-Leu-OMe) were heated to 120° C. in a water jet vacuum, stirring well, and stirred at this temperature for 2 hours. After cooling, 20 mL methanol was added to the slurry of solid matter, it was stirred briefly, filtered with suction, washed with a little methanol and dried with the oil pump. The diastereomers of cyclo-Met-Leu were separated on a silica gel column with n-butanol/acetic acid/water=4:1:1 (v/v/v). An isolated fraction from this was characterized as an example.

### 3-(R)-[2-(methylthio)ethyl]-6-(S)-(2-(methyl)propyl)-2,5-piperazinedione (cyclo-D-Met-L-Leu)

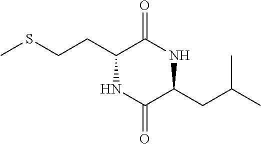

Final weight: 320 mg (59% of theoretical) as white solid.

¹H-NMR of 3-(R)-[2-(methylthio)ethyl]-6-(S)-(2-(methyl)propyl)-2,5-piperazinedione (cyclo-D-Met-L-Leu) (500 MHz, d₆-DMSO): δ=0.87 (d, ³J=6.6 Hz, 3H, CH(CH₃)(CH₃)); 0.88 (d, ³J=6.6 Hz, 3H, CH(CH₃)(CH₃)); 1.50-1.58 (m, 2H, SCHCH₂); 1.72-1.84 (m, 1H, CH(CH₃)₂); 1.88-2.02 (m, 2H, (CH₃)₂HCH₂); 2.04 (s, 3H, SCH₃); 2.44-2.58 (m, 2H, SCH₂); 3.68-3.74 (m, 1H, CH); 3.94-4.00 (m, 1H, CH); 8.11 (bs, 1H, NH); 8.19 (bs, 1H, NH)

¹³C-NMR of 3-(R)-[2-(methylthio)ethyl]-6-(S)-(2-(methyl)propyl)-2,5-piperazinedione (cyclo-D-Met-L-Leu) (125.8 MHz, d₆-DMSO): δ=14.40; 21.82; 22.77; 23.51; 28.51; 31.28; 41.92; 52.50; 52.90; 167.67; 168.82

### Example 6

### Synthesis of cyclic cyclo-Met-Phe dipeptides from DL-methionine methyl ester (DL-Met-OMe) and L-phenylalanine methyl ester (L-Phe-OMe) and isolation of a diastereomer 3-(R)-[2-(methylthio)ethyl]-6-(S)-(2-phenylmethyl)-2,5-piperazinedione (cyclo-D-Met-L-Phe)

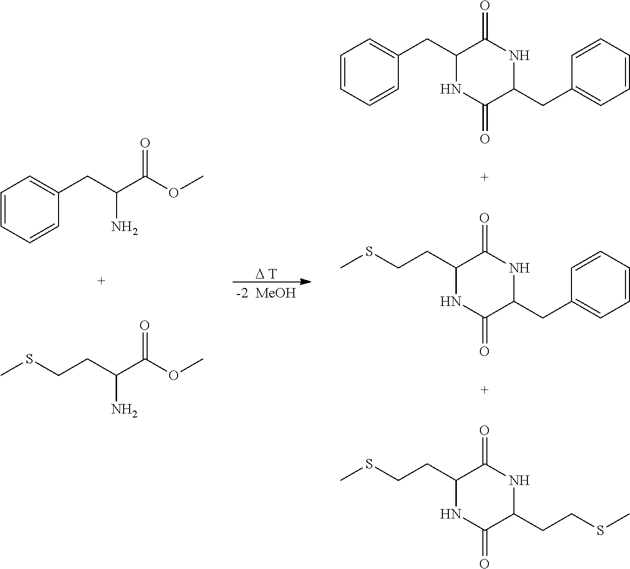

4.08 g (25 mmol) of DL-methionine methyl ester (DL-Met-OMe) and 4.48 g (25 mmol) of L-phenylalanine methyl ester (L-Phe-OMe) were heated to 120° C. in a water-jet vacuum, stirring well, and stirred at this temperature for 2.5 hours. After cooling, 30 mL methanol was added to the slurry of solid matter, it was stirred briefly, filtered with suction, washed with a little methanol and dried with the oil pump. The diastereomers of cyclo-Met-Phe were separated on a silica gel column with n-butanol/acetic acid/water=4:1:1 (v/v/v). An isolated fraction from this was characterized as an example.

### 3-(R)-[2-(methylthio)ethyl]-6-(S)-(2-phenylmethyl)-2,5-piperazinedione (cyclo-D-Met-L-Phe)

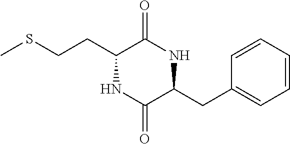

Final weight: 360 mg (77% of theoretical) as white solid.

¹H-NMR of 3-(R)-[2-(methylthio)ethyl]-6-(S)-(2-phenylmethyl)-2,5-piperazinedione (cyclo-D-Met-L-Phe) (500 MHz, d₆-DMSO): δ=1.76-1.82 (m, 2H, SCHCH₂); 1.97 (s, 3H, SCH₃); 2.30-2.46 (m, 2H, SCH₂); 2.89 (dd, 1H, ³J=4.8 Hz, ¹J=13.5 Hz, PhCH′H″); 3.05 (t, ³J=5.0 Hz, 1H, CH); 2.89 (dd, 1H, ³J=4.8 Hz, ¹J=13.5 Hz, PhCH′H″); 4.10-4.16 (m, 1H, CH); 7.14-7.30 (m, 5H, ph); 8.03 (bs, 1H, NH); 8.13 (bs, 1H, NH)

¹³C-NMR of 3-(R)-[2-(methylthio)ethyl]-6-(S)-(2-phenylmethyl)-2,5-piperazinedione (cyclo-D-Met-L-Phe) (125.8 MHz, d₆-DMSO): δ=14.38; 28.22; 31.56; 38.44; 52.16; 55.43; 126.64; 128.00; 129.97; 135.92; 167.24; 167.33

### Example 7

### Synthesis of cyclic cyclo-Met-Thr dipeptides from DL-methionine methyl ester (DL-Met-OMe) and L-threonine methyl ester (L-Thr-OMe) and isolation of a diastereomer 3-(R)-[2-(methylthio)ethyl]-6-(S)-(1-(R)-hydroxyethyl)-2,5-piperazinedione (cyclo-D-Met-L-Thr)

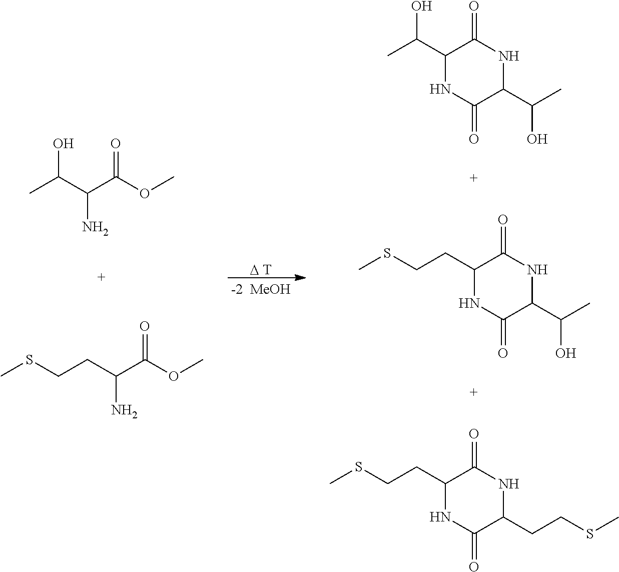

4.08 g (25 mmol) of DL-methionine methyl ester (DL-Met-OMe) and 3.33 g (25 mmol) of L-threonine methyl ester (L-Thr-OMe) were heated to 100° C. in a water jet vacuum, stirring well, and stirred at this temperature for 3 hours. After cooling, 150 mL methanol was added to the slurry of solid matter, it was stirred briefly, filtered with suction, washed with a little methanol and dried with the oil pump. The diastereomers of cyclo-Met-Thr were separated on a silica gel column with n-butanol/acetic acid/water=4:1:1 (v/v/v). An isolated fraction from this was characterized as an example.

### 3-(R)-[2-(methylthio)ethyl]-6-(S)-(1-(R)-hydroxyethyl)-2,5-piperazinedione (cyclo-D-Met-L-Thr)

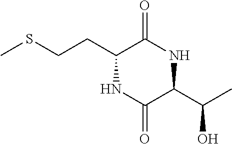

Final weight: 250 mg (54% of theoretical) as white solid.

¹H-NMR of 3-(R)-[2-(methylthio)ethyl]-6-(S)-(1-(R)-hydroxyethyl)-2,5-piperazinedione (cyclo-D-Met-L-Thr) (500 MHz, d₆-DMSO): δ=1.09 (d, 3H, ³J=6.3 Hz, CH(OH)CH₃); 1.86-2.02 (m, 2H, SCH₂CH₂); 2.04 (s, 3H, SCH₃); 2.42-2.60 (m, 2H, SCH₂); 2.42-2.46 (m, 1H, CH); 4.00-4.06 (m, 2H, CH, OCH₃); 4.98 (d, ³J=5.3 Hz, 1H, OH); 8.01 (bs, 1H, NH); 8.07 (bs, 1H, NH)

¹³C-NMR of 3-(R)-[2-(methylthio)ethyl]-6-(S)-(1-(R)-hydroxyethyl)-2,5-piperazinedione (cyclo-D-Met-L-Thr) (125.8 MHz, d₆-DMSO): δ=14.41; 19.88; 28.50; 30.77; 52.06; 60.93; 68.17; 168.61; 168.69

### Example 8

### Synthesis of cyclic cyclo-Met-Lys dipeptides from DL-methionine methyl ester (DL-Met-OMe) and L-lysine methyl ester (L-Lys-OMe) and isolation of a diastereomer 3-(S)-[2-(methylthio)ethyl]-6-(S)-(4-aminobutyl)-2,5-piperazinedione hydrochloride (cyclo-L-Met-L-Lys×HCl)

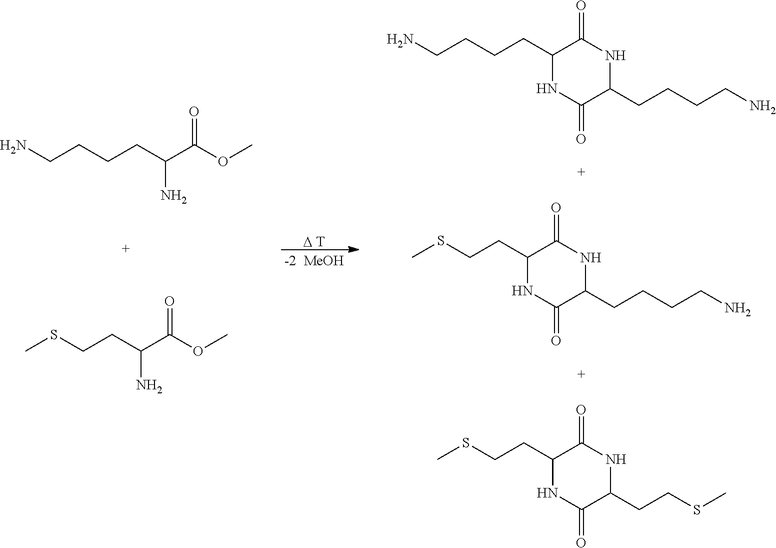

4.08 g (25 mmol) of DL-methionine methyl ester (DL-Met-OMe) and 3.33 g (25 mmol) of L-lysine methyl ester monohydrochloride (L-Lys-OMe×HCl) were heated to 130° C. in a water-jet vacuum, stirring well, and stirred at this temperature for 1.5 hours. After cooling, 150 mL methanol was added to the slurry of solid matter, it was stirred briefly, filtered with suction, washed with a little methanol and dried with the oil pump. The diastereomers of cyclo-Met-Lys were separated on a silica gel column with n-butanol/ethyl acetate/triethylamine=70:30:2 (v/v/v). As an example, an isolated fraction from this was dissolved in ethanolic HCl solution, concentrated by evaporation again, and characterized, after removing solvent residues with the vacuum pump.

### 3-(S)-[2-(methylthio)ethyl]-6-(S)-(4-aminobutyl)-2,5-piperazinedione hydrochloride (cyclo-L-Met-L-Lys×HCl)

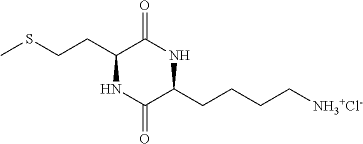

Final weight: 190 mg (40% of theoretical) as white solid.

¹H-NMR of 3-(S)-[2-(methylthio)ethyl]-6-(S)-(4-aminobutyl)-2,5-piperazinedione hydrochloride (cyclo-L-Met-L-Lys×HCl) (500 MHz, d₆-DMSO): δ=1.26-1.44 (m, 2H, CH₂); 1.50-1.60 (m, 2H, CH₂); 1.62-1.74 (m, 2H, CH₂); 1.82-1.92 (m, 2H, SCHCH₂); 2.04 (s, 3H, SCH₃); 2.50-2.60 (m, 2H, SCH₂); 2.70-2.78 (m, 2H, NCH₂); 3.82-3.88 (m, 1H, CH); 3.92-3.96 (m, 1H, CH); 8.01 (bs, 3H, NH₃⁺); 8.16 (bs, 1H, NH); 8.19 (bs, 1H, NH)

¹³C-NMR of 3-(S)-[2-(methylthio)ethyl]-6-(S)-(4-aminobutyl)-2,5-piperazinedione hydrochloride (cyclo-L-Met-L-Lys×HCl) (125.8 MHz, d₆-DMSO): δ=14.38; 21.02; 26.57; 28.77; 31.92; 32.48; 38.41; 52.93; 53.60; 167.66; 167.86

### Example 9

### Synthesis of cyclic cyclo-Met-Val dipeptides from DL-methionine methyl ester (DL-Met-OMe) and L-valine methyl ester (L-val-OMe) and isolation of a diastereomer 3-(R)-[2-(methylthio)ethyl]-6-(S)-(1-(methyl)ethyl)-2,5-piperazinedione (cyclo-D-Met-L-Val)

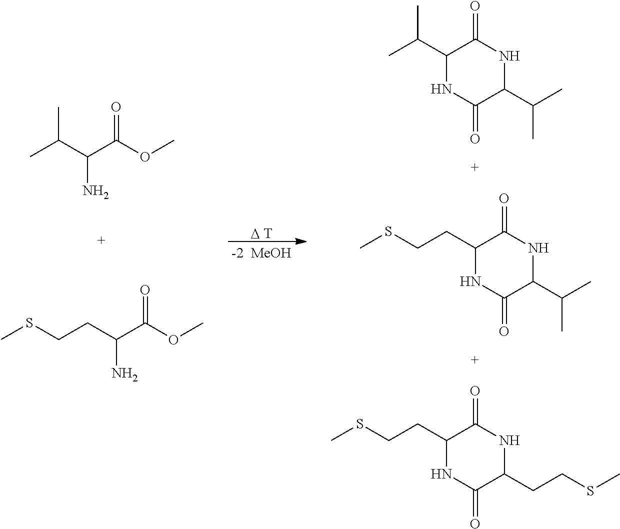

4.08 g (25 mmol) of DL-methionine methyl ester (DL-Met-OMe) and 3.28 g (25 mmol) of L-valine methyl ester (L-val-OMe) were heated to 120° C. in a water jet vacuum, stirring well, and stirred at this temperature for 2 hours. After cooling, 20 mL methanol was added to the slurry of solid matter, it was stirred briefly, filtered with suction, washed with a little methanol and dried with the oil pump. The diastereomers of cyclo-Met-Val were separated on a silica gel column with n-butanol/acetic acid/water=4:1:1 (v/v/v). An isolated fraction from this was characterized as an example.

### 3-(R)-[2-(methylthio)ethyl]-6-(S)-(1-(methyl)ethyl)-2,5-piperazinedione (cyclo-D-Met-L-Val)

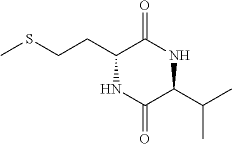

Final weight: 380 mg (82% of theoretical) as white solid.

¹H-NMR of 3-(R)-[2-(methylthio)ethyl]-6-(S)-(1-(methyl)ethyl)-2,5-piperazinedione (cyclo-D-Met-L-Val) (500 MHz, d₆-DMSO): δ=0.85 (d, ³J=7.0 Hz, 3H, CH(CH₃)(CH₃)); 0.93 (d, ³J=7.0 Hz, 3H, CH(CH₃)(CH₃)); 1.88-2.00 (m, 2H, SCHCH₂); 2.04 (s, 3H, SCH₃); 2.10-2.18 (m, 1H, CH(CH₃)₂); 2.42-2.58 (m, 2H, SCH₂); 3.58-3.62 (m, 1H, CH); 3.94-4.00 (m, 1H, CH); 8.11 (bs, 1H, NH); 8.13 (bs, 1H, NH)

¹³C-NMR of 3-(R)-[2-(methylthio)ethyl]-6-(S)-(1-(methyl)ethyl)-2,5-piperazinedione (cyclo-D-Met-L-Val) (125.8 MHz, d₆-DMSO): δ=14.42; 16.98; 18.32; 28.36; 31.71; 31.94; 52.40; 59.72; 167.53; 167.78

### Example 10

### Synthesis of cyclic cyclo-Met-Ile dipeptides from DL-methionine methyl ester (DL-Met-OMe) and L-isoleucine methyl ester (L-Ile-OMe) and isolation of a diastereomer 3-[2-(methylthio)ethyl]-6-(1-(methyl)propyl)-2,5-piperazinedione (cyclo-D-Met-L-Ile)

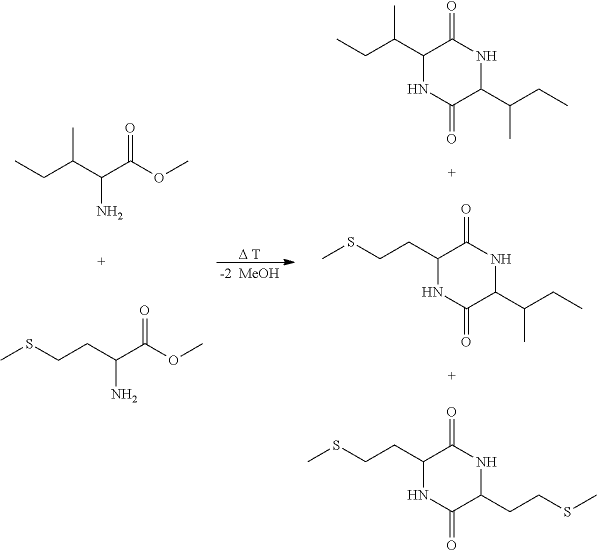

4.08 g (25 mmol) of DL-methionine methyl ester (DL-Met-OMe) and 3.63 g (25 mmol) of L-isoleucine methyl ester (L-Ile-OMe) were heated to 120° C. in a water jet vacuum, stirring well, and stirred at this temperature for 2 hours. After cooling, 20 mL methanol was added to the slurry of solid matter, it was stirred briefly, filtered with suction, washed with a little methanol and dried with the oil pump. The diastereomers of cyclo-Met-Ile were separated on a silica gel column with n-butanol/acetic acid/water=4:1:1 (v/v/v). An isolated fraction from this was characterized as an example.

### 3-[2-(methylthio)ethyl]-6-(1-(methyl)propyl)-2,5-piperazinedione (cyclo-D-Met-L-Ile)

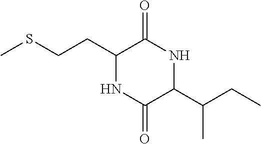

¹H-NMR of 3-[2-(methylthio)ethyl]-6-(1-(methyl)propyl)-2,5-piperazinedione (cyclo-D-Met-L-Ile) (500 MHz, d₆-DMSO): δ=0.85 (t, ³J=7.4 Hz, 3H, CH₂CH₃); 0.90 (d, ³J=7.4 Hz, 3H, CHCH₃); 1.10-1.50 (m, 2H, SCH₂CH₂); 1.80-1.90 (m, 1H, CH); 1.90-2.00 (m, 2H, CH₂); 2.04 (s, 3H, SCH₃); 2.42-2.58 (m, 2H, SCH₂); 3.64-3.68 (m, 1H, CH); 3.94-3.98 (m, 1H, CH); 8.08-8.16 (m, 2H, 2×NH)

¹³C-NMR of 3-[2-(methylthio)ethyl]-6-(1-(methyl)propyl)-2,5-piperazinedione (cyclo-D-Met-L-Ile) (125.8 MHz, d₆-DMSO+HCl): δ=12.02; 14.85; 15.27; 24.61; 28.74; 32.15; 39.90; 52.92; 59.34; 167.90; 168.10

### Example 11

In vitro digestion tests on 3,6-bis[2-(methylthio)ethyl]-2,5-piperazinedione (DD/LL-cyclo-Met-Met) with digestive enzymes from omnivorous mirror carp

a) Isolation of the Digestive Enzymes from Mirror Carp (Cyprinus carpio morpha noblis)

The digestive enzymes were isolated based on the method of EID and MATTY (Aquaculture 1989, 79, 111-119). For this, the intestine was removed from six one-year-old mirror carp (Cyprinus carpio morpha noblis), rinsed with water, cut open lengthwise and in each case the intestinal mucosa was scraped off. This was comminuted, together with crushed ice, in a mixer. The resultant suspension was treated with an ultrasonic rod, to disrupt any cells that were still intact. In order to separate the cell constituents and fat, the suspension was centrifuged for 30 minutes at 4° C., the homogenate was decanted off and sterilized with a trace of thimerosal. 49 ml of enzyme solution of the intestinal mucosa was obtained from 6 mirror carp. The solution was stored at 4° C. in the dark.

### b) Procedure for the In Vitro Digestion Studies

DD/LL-cyclo-Met-Met was taken up in TRIS/HCl buffer solution and the enzyme solution was added to it. As comparison and for assessing the purely chemical cleavage rate, in each case a blank was prepared without enzyme solution (see Table 1). A sample was taken from time to time and its composition was detected and quantified using a calibrated HPLC. The conversion was determined as the quotient of the content of methionine or methionylmethionine (Met-Met) and the content of DD/LL-cyclo-Met-Met (see FIG. 4). In the blank there was hardly any reaction of DD/LL-cyclo-Met-Met to the dipeptide DD/LL-Met-Met or DL-methionine.

TABLE 1
Cleavage of DD/LL-cyclo-Met-Met

| | | Sample | | Blank | |
|---|---|---|---|---|---|
| Charge | Substrate | 0.15 | mmol | 0.15 | mmol |
| | (DD/LL-cyclo- | | | | |
| | Met-Met) | | | | |
| | TRIS/HCl buffer solution, | 90.9 | ml | 95.0 | ml |
| | pH 9.5 | | | | |
| | bile | 5.00 | ml | 5.00 | ml |

| | | | | |
|---|---|---|---|---|
| Reaction | Enzyme solution | 4.1 | ml | — |

| | | | |
|---|---|---|---|
| start | (\hat{=}50% carp solution) | | |
| Reaction | | 37° C. | 37° C. |

| | |
|---|---|
| Stopping | 1.0 ml of reaction solution was taken up in 5.0 ml of |
| the | a 1:1 (v/v) mixture of 10% H₃PO₄ solution and acetonitrile, |
| reaction | stirred for 20min and filtered on a 20 μm syringe filter. |

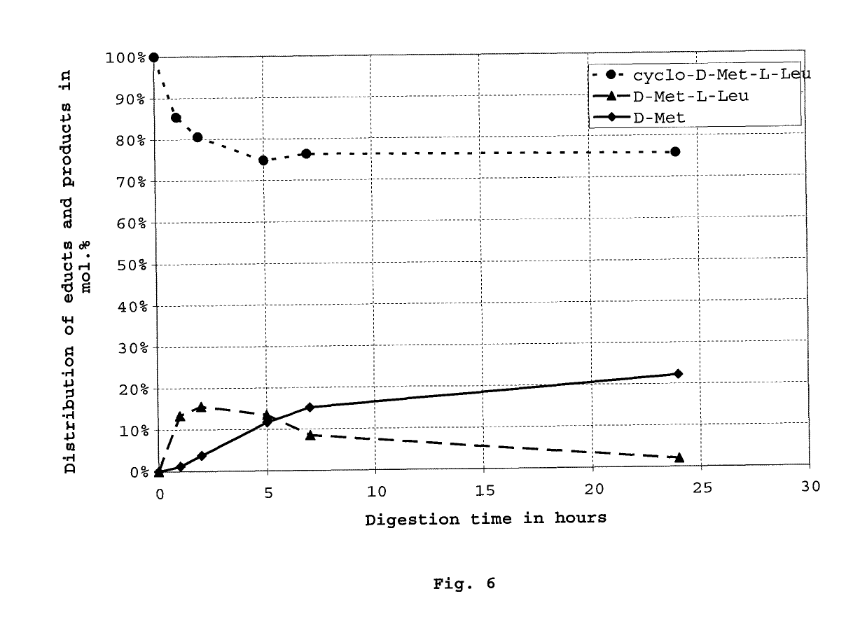

The test from Example 11b) was carried out similarly, with the cyclic dipeptides cyclo-L-His-L-His and cyclo-D-Met-L-Leu (see FIGS. 5 and 6).

### Example 12

### In vitro digestion tests on DD/LL/meso-cyclo-Met-Met with digestive enzymes from carnivorous rainbow trout

a) Isolation of the Digestive Enzymes from Rainbow Trout (Oncorhynchus mykiss)

The digestive enzymes were isolated based on the method of EID and MATTY (Aquaculture 1989, 79, 111-119). For this, the intestine was removed from five one-year-old rainbow trout (Oncorhynchus mykiss) and processed as described in Example 11. 56 ml of enzyme solution of the intestinal mucosa was obtained from 5 rainbow trout. The solution was stored in the dark at 4° C.

### b) Procedure for the In Vitro Digestion Studies

The in vitro studies were carried out as in Example 11. The conversion was determined as the quotient of the content of methionine or methionylmethionine (Met-Met) and the content of cyclo-Met-Met (see FIG. 3). In the blank, there was hardly any conversion of DD/LL/meso-cyclo-Met-Met to the dipeptide DD/LL/meso-Met-Met or DL-methionine.

TABLE 2
Cleavage of DD/LL-cyclo-Met-Met

| | | Sample | | Blank | |
|---|---|---|---|---|---|
| Charge | Substrate | 0.10 | mmol | 0.10 | mmol |
| | (DD/LL/meso- | | | | |
| | cyclo-Met-Met) | | | | |
| | TRIS/HCl buffer solution, | 5.0 | ml | 10.3 | ml |
| | pH 9.5 | | | | |
| | Bile | 1.0 | ml | 0.0 | ml |
| | Liver Homogenate | 1.5 | mL | 0.0 | ml |

| | | | | |
|---|---|---|---|---|
| Reaction | Enzyme solution | 2.8 | ml | — |

| | | | |
|---|---|---|---|
| start | (\hat{=}25% trout solution) | | |
| Reaction | | 37° C. | 37° C. |

| | |
|---|---|
| Stopping | 1.0 ml of reaction solution was taken up in 5.0 ml of |
| the | a 1:1 (v/v) mixture of 10% H₃PO₄ solution and acetonitrile, |
| reaction | stirred for 20 min and filtered on a 20 μm syringe filter. |

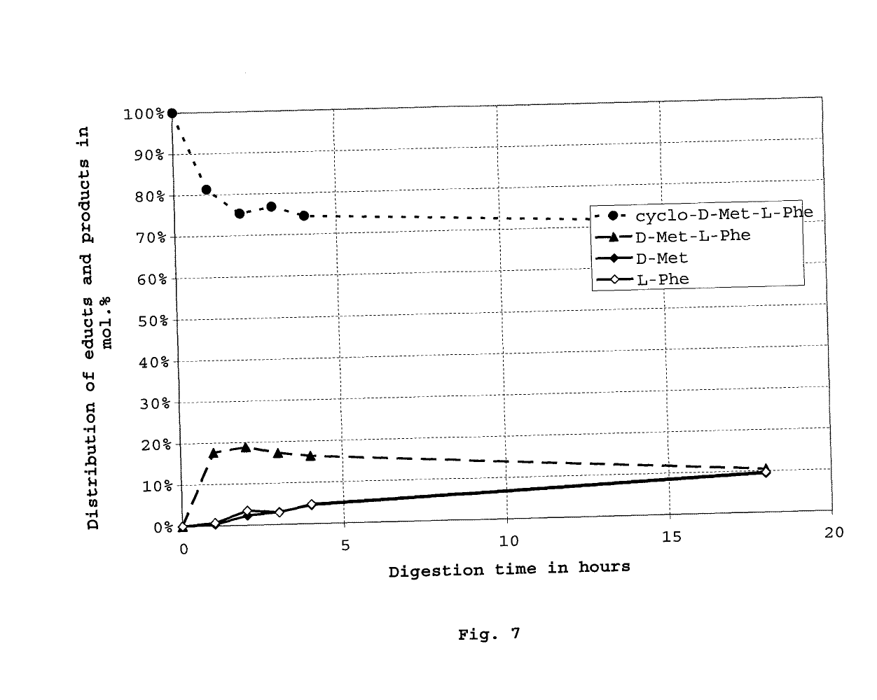

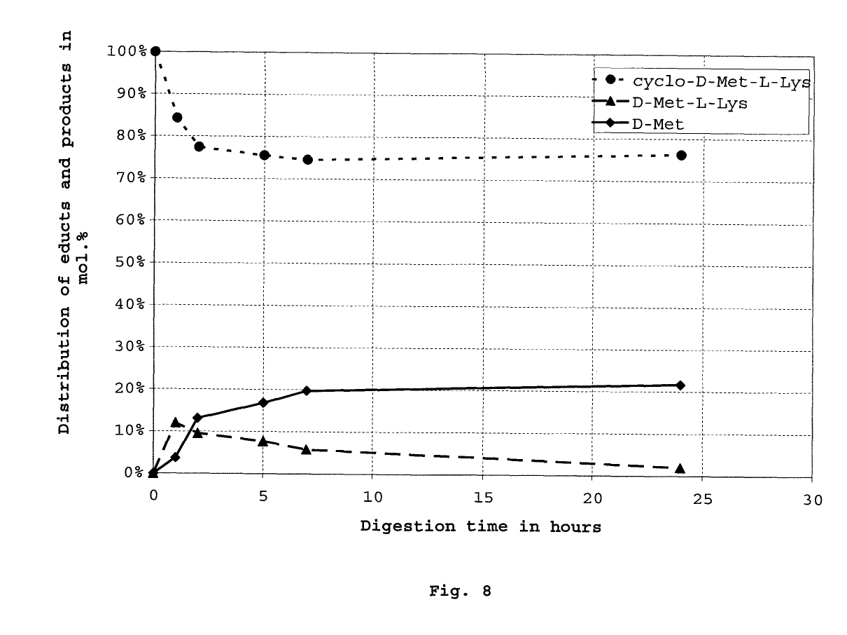

The test from Example 12b) was carried out similarly, with the cyclic dipeptides cyclo-D-Met-L-Phe and cyclo-D-Met-L-Lys (see FIGS. 7 and 8).

### Example 13

### In vitro digestion tests on cyclo-D-Met-L-Thr with digestive enzymes from omnivorous shrimps

a) Isolation of the Digestive Enzymes from Whiteleg Shrimps (Litopenaeus vannamei)

The digestive enzymes were isolated based on the method of Ezquerra and Garcia-Carreno (J. Food Biochem. 1999, 23, 59-74). For this, the hepatopancreas was removed from 2.1 kilograms (57 animals) of whiteleg shrimps (Litopenaeus vannamei) and comminuted together with crushed ice in a mixer. Further processing was carried out as in Example 11. 74 ml of enzyme solution of the intestinal mucosa was obtained from 57 whiteleg shrimps. The solution was stored in the dark at 4° C.

### b) Procedure for the In Vitro Digestion Studies

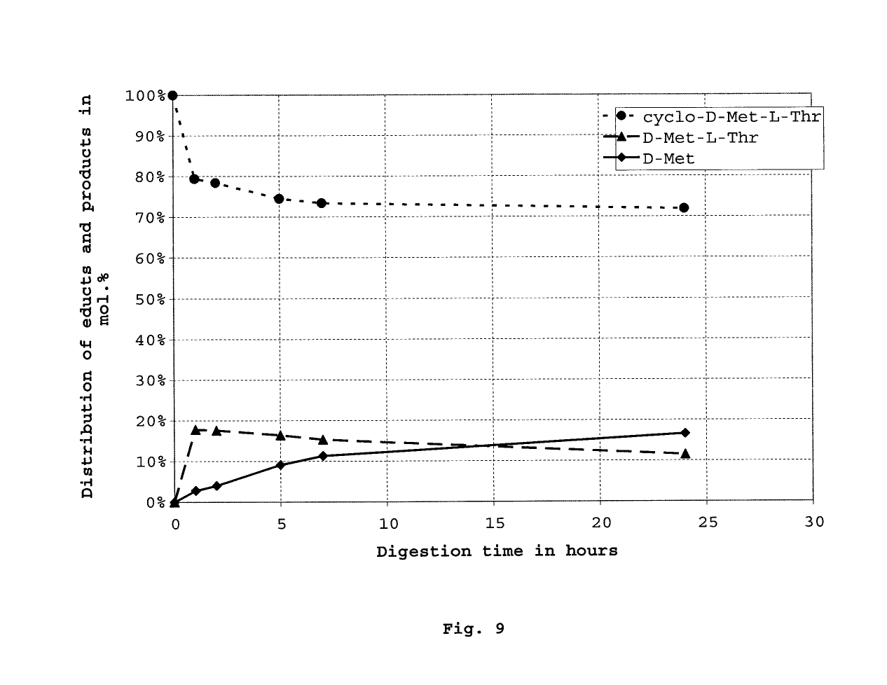

The in vitro studies were carried out as in Example 11. The conversion was determined as the quotient of the content of methionine or D-Met-L-Thr and the content of cyclo-D-Met-L-Thr (see FIG. 9). In the blank there was hardly any conversion of cyclo-D-Met-L-Thr to the dipeptide D-Met-L-Thr or to the free amino acids.

TABLE 3
Cleavage of cyclo-D-Met-L-Thr

| | | Sample | | Blank | |
|---|---|---|---|---|---|
| Charge | Substrate | 0.15 | mmol | 0.15 | mmol |
| | (cyclo-D-Met-L-Thr) | | | | |
| | TRIS/HCl buffer solution, | 8.5 | ml | 14.0 | ml |

| | pH 9.5 | | | |
|---|---|---|---|---|
| Reaction start | Enzyme solution | 6.5 | ml | — |

| | | | |
|---|---|---|---|
| | (\hat{=}5 shrimps) | | |
| Reaction | | 37° C. | 37° C. |

| | |
|---|---|
| Stopping the | 0.2 ml of reaction solution was taken up |
| reaction | in 9.8 ml of 10% H₃PO₄ solution. |

1. A feed additive, comprising:
at least one diketopiperazine of formula (IV) or a salt thereof: 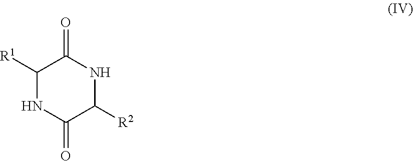
wherein
R¹ and R² are each, independently, an amino acid residue R selected from the group consisting of methionine (R=—(CH₂)₂SCH₃), lysine (R=—(CH₂)₄NH₂), threonine (R=—CH(OH)(CH₃)), tryptophan (R=-indolyl), histidine (R=-imidazoyl), valine (R=—CH(CH₃)₂), leucine (R=—CH₂CH((CH₃)₂), isoleucine (R=—CH(CH₃)CH₂CH₃), phenylalanine (R=—CH₂Ph), arginine (R=—(CH₂)₃NHC(═NH)NH₂), cysteine (R=—CH₂SH); or
at least one compound of formula (V) or a salt thereof: 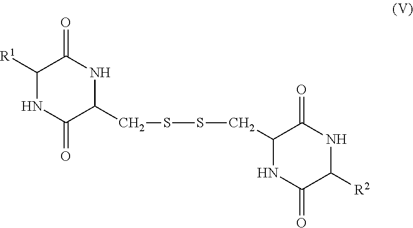
wherein R¹ and R² are as defined above.

2. The feed additive according to claim 1, wherein
R¹ or R² is a methionyl residue (R=—(CH₂)₂SCH₃), and
a configuration of the structure is at least one selected from the group consisting of a DD-, a LL-, a LD- and a DL-configuration.

3. The feed additive according to claim 1, wherein
a configuration of the diketopiperazine of formula (IV) is at least one selected from the group consisting of a cyclo-D-EAA-D-EAA, a cyclo-L-EAA-D-EAA, a cyclo-D-EAA-L-EAA and a cyclo-L-EAA-L-EAA, and
EAA is an amino acid selected from the group consisting of methionine, lysine, threonine, tryptophan, histidine, valine, leucine, isoleucine, phenylalanine, arginine, cysteine and cystine.

4. The feed additive according to claim 3, wherein the diketopiperazine of formula (IV) is a diastereomeric mixture of cyclo-DL-EAA-DL-EAA.

5. The feed additive according to claim 1, wherein
R¹ and R² are both a methionyl residue (R=—(CH₂)₂SCH₃), and
the diketopiperazine of formula (IV) is in the DD-, LL-, DL- or LD-configuration or a mixture thereof,
with the proviso that the diketopiperazine with the LL-configuration is only present in a mixture with at least one other configuration.

6. The feed additive according to claim 5, wherein
the diketopiperazine of formula (IV) is a diastereomeric mixture comprising DD/LL-cyclo-Met-Met and meso-cyclo-Met-Met, where Met is methionine.

7. A feed mixture, comprising at least one feed additive according to claim 1.

8. The feed mixture according to claim 7, further comprising: at least one substance selected from the group consisting of DL-methionine, L-EAA, DL-EAA, DD/LL/DL/LD-methionyl-EAA, DD/LL/DL/LD-EAA-methionine, DD/LL-methionyl-EAA, DD/LL-EAA-methionine, D-methionyl-L-EAA, L-methionyl-L-EAA, D-methionyl-D-EAA, L-methionyl-D-EAA, D-EAA-L-methionine, L-EAA-L-methionine, D-EAA-D-methionine and L-EAA-D-methionine.

9. The feed mixture according to claim 7, further comprising:
DL-methionine; and
at least one substance selected from the group consisting of L-EAA, DL-EAA, DD/LL/DL/LD-methionyl-EAA, DD/LL/DL/LD-EAA-methionine, DD/LL-methionyl-EAA, DD/LL-EAA-methionine, D-methionyl-L-EAA, L-methionyl-L-EAA, D-methionyl-D-EAA, L-methionyl-D-EAA, D-EAA-L-methionine, L-EAA-L-methionine, D-EAA-D-methionine and L-EAA-D-methionine;
wherein a proportion of DL-methionine is from 0.01 to 90 wt. %.

10. The feed mixture according to claim 7, further comprising:
DL-methionine;
a L-EAA and
at least one substance selected from the group consisting of DL-EAA, DD/LL/DL/LD-methionyl-EAA, DD/LL/DL/LD-EAA-methionine, DD/LL-methionyl-EAA, DD/LL-EAA-methionine, D-methionyl-L-EAA, L-methionyl-L-EAA, D-methionyl-D-EAA, L-methionyl-D-EAA, D-EAA-L-methionine, L-EAA-L-methionine, D-EAA-D-methionine and L-EAA-D-methionine;
wherein a proportion of the DL-methionine is from 0.01 to 90 wt. % and a proportion of the L-EAA is from 0.01 to 90 wt. %.

11. A diketopiperazine or a salt thereof, of formula (IV): 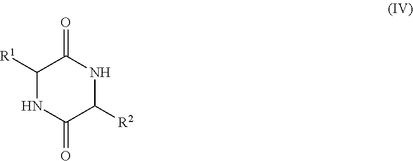
wherein
R¹ and R² are each, independently, an amino acid residue selected from the group consisting of methionine (R^{1/2}=—(CH₂)₂SCH₃), lysine (R^{1/2}=—(CH₂)₄NH₂), threonine (R^{1/2}=—CH(OH)(CH₃)), tryptophan (R^{1/2}=-indolyl), histidine (R^{1/2}=-imidazoyl), valine (R^{1/2}=—CH(CH₃)₂), leucine (R^{1/2}=—CH₂CH((CH₃)₂), isoleucine (R^{1/2}=—CH(CH₃)CH₂CH₃), phenylalanine (R^{1/2}=—CH₂Ph), arginine (R^{1/2}=—(CH₂)₃NHC(═NH)NH₂), cysteine (R^{1/2}=—CH₂SH),
with the proviso that when R¹ and R² are both —(CH₂)₂SCH₃, the diketopiperazine is not exclusively in the form of cyclo-L-Met-L-Met; or
a compound of formula (V) or a salt thereof: 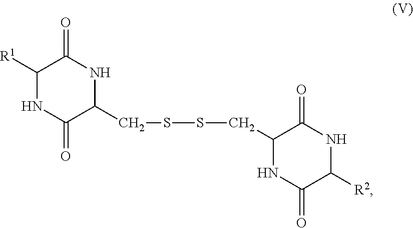
wherein R¹ and R² are as defined above.

12. The diketopiperazine according to claim 11, wherein a configuration of the diketopiperazine of formula (IV) is at least one selected from the group consisting of cyclo-D-EAA-D-EAA, cyclo-L-EAA-D-EAA, cyclo-D-EAA-L-EAA, cyclo-L-EAA-L-EAA,
wherein EAA is an amino acid selected from the group consisting of methionine, lysine, threonine, tryptophan, histidine, valine, leucine, isoleucine, phenylalanine, arginine, cysteine and cystine.

13. The diketopiperazine according to claim 11, wherein a configuration of the diketopiperazine of formula (IV) is a diastereomeric mixture of cyclo-DL-EAA-DL-EAA.

14. The diketopiperazine according to claim 11, wherein R¹ and R² are both a methionyl residue (R=—(CH₂)₂SCH₃), and
the diketopiperazine of formula (IV) is in the DD-, LL-, DL- or LD-configuration or a mixture thereof, with the proviso that the diketopiperazine with the LL configuration is only present mixed with other configurations.

15. The diketopiperazine according to claim 14, wherein the diketopiperazine of formula (IV) is a mixture of DD/LL-cyclo-Met-Met and meso-cyclo-Met-Met.

16. A feed additive for ruminants, fresh or salt water fishes and crustaceans comprising the diketopiperazine or salt thereof according to claim 11.

17. A method of production of a diketopiperazine of formula (IV) or a salt thereof: 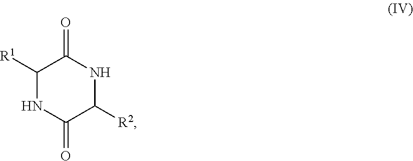
or a compound of formula (V) or a salt thereof: 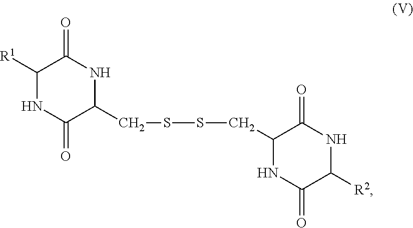
comprising reacting one or more amino acid esters of formula (III) in substance, and in absence of a solvent, to obtain the diketopiperazine of formula (IV) or (V): 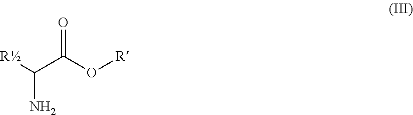
wherein R¹ and R² are each independently at least one selected from the group consisting of 2-(methylthio)ethyl)-, 1-methylethyl-, 2-methylpropyl-, (1S)-1-methylpropyl-, (1R)-1-hydroxyethyl-, 4-aminobutyl-, 3-[(aminoiminomethyl)-amino]-propyl-, benzyl-, (1H-imidazol-4-yl)methyl-, (1H-indol-3-yl)methyl-, mercaptomethyl- and —CH₂—S—S—CH₂—CH(NH₂)COOR′
wherein optionally, R¹ is equal to R²;
and wherein R′ is a linear or branched aliphatic residue or aromatic residue and different R′ can be present in different amino acid ester molecules.

18. The method according to claim 17, wherein the amino acid ester of formula (III) is obtained by esterification of an amino acid of formula (I): 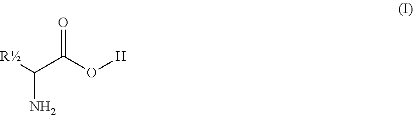
wherein R^{1/2} is —CH(NH₂)—COOH or cystine with a compound of formula (II):
R′—OH (II).

19. The method according to claim 18, wherein the esterification is carried out in the presence of a strong acid.

20. The method according to claim 19, wherein the strong acid is HCl or H₂SO₄.

21. The method according to claim 17, wherein R′ is selected from the group consisting of methyl, ethyl, n-propyl, iso-propyl, n-butyl, iso-butyl, sec-butyl, and benzyl.

22. The method according to claim 17, wherein a temperature of the reaction of the amino acid ester to form the diketopiperazine is from 30 to 220° C.

23. The method according to claim 17, further comprising removing the compound of formula II by distillation.

24. The method according to claim 17, wherein the reaction of the amino acid ester to obtain the diketopiperazine takes place in absence of a transamidation catalyst.

25. The method according to claim 17, wherein a purity of the obtained diketopiperazine is greater than 50 wt. % as determined according to HPLC.

26. The method according to claim 17, further comprising:
recovering amino acid ester not reacted; and
recycling the recovered amino acid ester to the process.

27. The method according to claim 17, further comprising:
recovering the compound of formula (III); and
recycling the compound to a process for preparing the ester of formula (I).

28. Method according to claim 17, wherein a compound of formula R′—OH not completely converted in the esterification of the amino acid to the amino acid ester and/or obtained again in the conversion of the amino acid ester to the diketopiperazine is recovered and returned to the process.
